(19) United States
(12) Patent Application Publication
(10) Pub. No.: US 2026/0083636 A1
(43) Pub. Date: Mar. 26, 2026

(54) PRE-FILLED MULTI-FLUID MEDICAL DELIVERY ASSEMBLY

(71) Applicant: Koska Family Limited, East Sussex (GB)

(72) Inventors: Jeff PRICE, Windermere, FL (US); Daniel AMENT, Denver, CO (US); Jordan Michael YOST, Erie, PA (US); Jason Christopher BRYANT, Erie, PA (US)

(21) Appl. No.: 19/003,255

(22) Filed: Dec. 27, 2024

Related U.S. Application Data
(63) Continuation of application No. PCT/US24/48022, filed on Sep. 23, 2024.

(51) Int. Cl. A61J 1/14; A61J 1/20

(52) U.S. Cl. CPC A61J 1/1493; A61J 1/2013; A61J 1/2096

(57) ABSTRACT

A medical system, comprises a reservoir module defining a longitudinal axis, including a Blow-Fill-Seal (BFS) vial having a compressible body and a neck extending from the compressible main body. The compressible main body defines a first reservoir having a first fluid agent therein and a second reservoir having a second fluid agent therein. A first penetrable seal within the neck seals the first fluid agent and a second penetrable seal within the neck for sealing the second fluid agent.

### CROSS REFERENCE TO RELATED APPLICATION(S)

This application is a continuation of PCT Application Serial No. PCT/US24/48022, filed Sep. 23, 2024, and titled “PRE-FILLED MULTI-FLUID MEDICAL DELIVERY ASSEMBLY”, which claims the benefit and priority under 35 U.S.C. § 119(e) to, U.S. provisional Application Ser. No. 63/540,057, filed Sep. 23, 2023, the entire contents of the disclosure being incorporated herein.

### STATEMENT REGARDING FEDERALLY SPONSORED RESEARCH

This invention was made with government support under Award No. 75A50120C00007, awarded by the Department of Health and Human Services (HHS). The government has certain rights in the invention.

### BACKGROUND

Disposable injector devices for administrating a medical agent such as a vaccine, medicament, drug etc. typically include a single chamber for storing the medical agent which is distributed to the patient through a needle of the injector device. However, some medical agents may include multiple medicaments which require independent storage (and sometimes subsequent mixing) prior to delivery to the subject as a single dose. Dual chamber injection devices for separately storing multiple liquid agents until time of administration and/or for combined delivery of the agents to the subject are known, but are complex, not cost effective to produce and/or not feasible to manufacture in large quantities in a short amount of time if demand unexpectedly peaks.

In addition, some fluid agents stored in such devices have a defined shelf life. Because such devices are not able to be produced in large quantities at scale to meet unexpected peak demand, providers of such devices are put in a position of paying to pre-order and have manufactured inventories of such devices that sometimes need to be discarded due to expiration dates. Accordingly, these devices are not only not cost-effective to manufacture in the first place, they are further subject to waste.

Previous attempts at providing single-use or disposable injection devices to remedy such problems in the industry have achieved measurable success but have failed to adequately remedy the existing problems.

### SUMMARY

One or more embodiments of the disclosed subject matter provide systems, assemblies, kits, and methods for medical delivery of liquid agents to a patient. Using a blow-fill-seal (BFS) fabrication technique, separate BFS vials (such as bottles, or other components) are pre-filled with respective fluid (e.g., liquid) agents, which can be maintained separate, for example, until ready for use or administration to a patient.

In some embodiments, a medical system comprises a reservoir module defining a longitudinal axis. The reservoir module includes a Blow-Fill-Seal (BFS) vial having a compressible body and a neck extending from the compressible main body. The compressible body defines a first reservoir having a first fluid agent therein and a second reservoir having a second fluid agent therein. The vial further includes a first penetrable seal within the neck for sealing the first fluid agent and a second penetrable seal within the neck for sealing the second fluid agent. A delivery module including an administration member is configured to penetrate the first and second penetrable seals to access the first and second fluid agents for administration to a patient.

In embodiments, the neck defines a first neck passage in fluid communication with the first reservoir and terminating at the first penetrable seal and a second neck passage in fluid communication with the second reservoir and terminating at the second penetrable seal. In some embodiments, the first neck passage extends beyond the second neck passage. The first penetrable seal is in general longitudinal alignment with the second penetrable seal.

In embodiments, the system comprises a delivery module couplable to the reservoir module. The delivery module includes a hub for coupling to the vial of the reservoir module and an administration member coupled to the hub for distribution of the first fluid agent and the second fluid agent.

In embodiments, the administration member includes a needle. The needle has a seal penetrating end configured to penetrate the first and second penetrable seals of the vial upon mounting of the hub of the delivery module to the vial of the reservoir module. The needle may define one or more needle passages for passage of the first fluid agent and the second fluid agent. In some embodiments, the needle defines a first needle port and a second needle port where the first and second needle ports are in fluid communication with the one or more needle passages of the needle. According to some embodiments, when the delivery module is coupled to the reservoir module, the first needle port is in fluid communication with the first neck passage and the first reservoir, and the second needle port is in fluid communication with the second neck passage and the second reservoir.

In some embodiments, the vial of the reservoir module defines a fluid chamber in fluid communication with the first reservoir and with the second reservoir upon penetration of the seal penetrating end of the needle through the first penetrable seal and the second penetrable seal to enable mixing of the first fluid agent and the second fluid agent. In some embodiments, the first and second fluids may not be mixed in any meaningful sense but may rather be delivered into the patient at essentially the same time via the same needle that is operable to penetrate both fluid chambers during an activation process.

In embodiments, the needle includes a first inner needle and a second outer needle disposed about the first inner needle. The first inner needle defines a first needle passage, and the first inner needle and the second outer needle defining a second needle passage therebetween.

In some embodiments, the neck of the vial of the reservoir module includes a connection interface configured for at least partial reception within the hub of the delivery module.

In accordance with another illustrative embodiment, a pre-filled medical delivery assembly comprises a reservoir module comprising a blow-fill-seal (BFS) vial including a first vial component having a first penetrable seal and a first reservoir with a first fluid agent therein and a second vial component having a second penetrable seal and a second reservoir with a second fluid agent therein, and a delivery module couplable to the BFS vial. The delivery module includes a connector body for at least partially receiving the BFS vial and an administration member mounted to the connector body. The administration member includes a vial end and a patient end. The vial end defines a first port and a second port longitudinally spaced relative to the first port. Upon coupling of the BFS vial and the delivery module, the vial end of the administration member penetrates the first penetrable seal and the second penetrable seal in succession whereby the first port of the administration member is in fluid communication with the first fluid reservoir of the first vial component and the second port of the administration member is in fluid communication with the second fluid reservoir of the second vial component to permit passage of the first and second fluid agents through the administration member.

In embodiments, the first vial component defines a first chamber in fluid communication with the first fluid reservoir and the second vial component defines a second chamber in fluid communication with the first fluid reservoir. The first and second ports of the administration member are disposed within respective first and second chambers upon coupling of the BFS vial relative to the delivery module.

In some embodiments, the first and second ports of the administration member are side ports.

In certain embodiments, the first side port has an opening defining a first internal dimension and the second side port has an opening defining a second internal dimension different form the first internal dimension.

In other embodiments, the connector body includes a pair of locking arms with locking detents. The locking detents are received within cooperating locking recesses of the BFS vial upon coupling of the BFS vial relative to the delivery module to secure the BFS vial to the delivery module.

In certain embodiments, a removable protective cover is disposed over the administration member.

In embodiments, a pivoting shield is pivotally mounted relative to the connector body of the delivery module, the pivoting shield pivotal to selectively cover the administration member.

In other embodiments, the pivoting shield includes a latch to secure the pivoting shield to the administration member.

In some embodiments the first and second fluid agents are different.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Where applicable, some elements may be simplified or otherwise not illustrated in order to assist in the illustration and description of underlying features. For example, in some figures, some components have been illustrated using a partial or cutaway view in order to illustrate internal interaction of components. Throughout the figures, like reference numerals denote like elements. An understanding of embodiments described herein and many of the attendant advantages thereof may be readily obtained by reference to the following detailed description when considered with the accompanying drawings, wherein:

FIG. 1 is a perspective view of a pre-filled medical fluid system illustrating a reservoir module and a delivery module, according to one or more illustrative embodiments of the disclosed subject matter;

FIG. 2 is a side elevation view of a vial of a reservoir module of a pre-filled medical fluid system, according to one or more illustrative embodiments of the disclosed subject matter;

FIG. 3 is a side cross-sectional view of a vial of a reservoir module of a pre-filled medical fluid system, according to one or more illustrative embodiments of the disclosed subject matter;

FIG. 4 is an enlarged view of the area of detail identified in FIG. 3 according to one or more illustrative embodiments of the disclosed subject matter;

FIGS. 5A-5C are perspective, side elevation and cross-sectional views of a hub connector of a delivery module of a pre-filled medical fluid system, according to one or more illustrative embodiments of the disclosed subject matter;

FIGS. 6A-6D are perspective, side elevation and first and second isolated views of an administration member of a delivery module of a pre-filled medical fluid system, according to one or more illustrative embodiments of the disclosed subject matter;

FIGS. 7A-7C are perspective and first and second side elevation views of a protective cover of a delivery module of a pre-filled medical fluid system, according to one or more illustrative embodiments of the disclosed subject matter;

FIGS. 8A-8B are perspective and side cross-sectional views of a delivery module of a pre-filled medical fluid system, according to one or more illustrative embodiments of the disclosed subject matter;

FIGS. 9A-9C are perspective, side cross-sectional and isolated views of a delivery module coupled to a reservoir module of a pre-filled fluid medical system, according to one or more illustrative embodiments of the disclosed subject matter;

FIGS. 10A and 10B are partial perspective and cross-sectional views of an administration member of a delivery module of a pre-filled medical fluid system, according to one or more illustrative embodiments of the disclosed subject matter;

FIG. 11 is a perspective view of a pre-filled multi-fluid medical delivery assembly, according to one or more illustrative embodiments of the disclosed subject matter;

FIG. 12 is an exploded perspective view of the pre-filled multi-fluid medical delivery assembly illustrating the reservoir module and the delivery module, according to one or more illustrative embodiments of the disclosed subject matter;

FIG. 13 is a side elevation view of the reservoir module of the pre-filled multi-fluid medical delivery assembly, according to one or more illustrative embodiments of the disclosed subject matter;

FIG. 14 is a side cross-sectional view of the reservoir module of the pre-filled multi-fluid medical delivery assembly, according to one or more illustrative embodiments of the disclosed subject matter;

FIG. 15 is a side elevation view of the delivery module of the pre-filled multi-fluid medical delivery assembly, according to one or more illustrative embodiments of the disclosed subject matter;

FIG. 16 is a side cross-sectional view of the delivery module of the pre-filled multi-fluid medical delivery assembly, according to one or more illustrative embodiments of the disclosed subject matter;

FIG. 17 is an exploded perspective view of the delivery module illustrating the delivery connector, the cover and the pivoting shield of the pre-filled multi-fluid medical delivery assembly, according to one or more illustrative embodiments of the disclosed subject matter;

FIG. 18 is an enlarged isolated view of the area of detail identified in FIG. 12, according to one or more illustrative embodiments of the disclosed subject matter;

FIG. 19 is a perspective view of the pivoting shield of the delivery module of the pre-filled multi-fluid medical delivery assembly, according to one or more illustrative embodiments of the disclosed subject matter;

FIG. 20 is a cross-sectional view of the pivoting shield taken along the lines 20-20 of FIG. 19, according to one or more illustrative embodiments of the disclosed subject matter;

FIGS. 21 and 22 are views illustrating coupling of the delivery module and the reservoir module of the pre-filled multi-fluid medical delivery assembly, according to one or more illustrative embodiments of the disclosed subject matter;

FIG. 23 is an enlarged isolated view of the area of detail identified in FIG. 22, according to one or more illustrative embodiments of the disclosed subject matter;

FIG. 24 is a side cross-sectional view illustrating use of the pre-filled multi-fluid medical delivery assembly in distributing fluid agent(s) to the patient, according to one or more illustrative embodiments of the disclosed subject matter;

FIG. 25 is a cross-sectional view similar to the view of FIG. 20 illustrating the locking latch secured relative to the administration member of the pre-filled multi-fluid medical delivery assembly, according to one or more illustrative embodiments of the disclosed subject matter;

FIGS. 26 and 27 are perspective and side cross-sectional views of a reservoir module of the pre-filled multi-fluid medical delivery assembly, according to one or more illustrative embodiments of the disclosed subject matter;

FIGS. 28 and 29 are perspective and side cross-sectional views of a reservoir module of the pre-filled multi-fluid medical delivery assembly, according to one or more illustrative embodiments of the disclosed subject matter;

FIG. 30 is a cross-sectional view illustrating the first and second penetrable seals of a reservoir module of the pre-filled multi-fluid medical delivery assembly, according to one or more illustrative embodiments of the disclosed subject matter; and

FIGS. 31 and 32 are graphs representing various gauge needles, delivery times and delivery forces for distribution of the fluid agents from the reservoir modules of the pre-filled multi-fluid medical delivery assembly, according to one or more illustrative embodiments of the disclosed subject matter.

### DETAILED DESCRIPTION

### 1. Introduction

Described herein are systems, assemblies, kits, and methods for medical delivery of multiple fluid (e.g., liquid) agents (e.g., at least two) to a patient (e.g., human or animal) from pre-filled (and, in some embodiments, field-assembled or assembled at the point-of-use) modules. In some embodiments, at least two fluid agents are sealed in separate reservoirs of a reservoir module and maintained separate from each other until a time when it is desirable to mix the fluid agents (e.g., at the time of, or prior to use and/or administration to the patient). The fluid agents can be any type of agent to be injected into or otherwise delivered to a patient and capable of producing a therapeutic effect, either alone or in combination with an active ingredient. Accordingly, the fluid agents can include, but are not limited to, separate vaccines, drugs, medicaments, diluents, active ingredients, etc. that are desirable to combine for use and/or common administration to the patient. For example, in some embodiments, the combination of the fluid agents can form a multi-fluid agent, solution, mixture, suspension, etc. Alternatively, or additionally, the fluid agents can be separate components that, when combined, form a vaccine, drug, medicament, etc. For example, in some embodiments, the combination of the fluid agents can comprise a single dose of a therapeutic agent (e.g., vaccine, drug, medicament, etc.). In some embodiments, one or more of the fluid agents in each reservoir can be tracked, monitored, checked for compatibility, etc., such as by utilization of electronic data storage devices (not shown) coupled to the various modules or components of the system.

In some embodiments, the reservoir module may comprise a blow-fill-seal (BFS) vial or container that includes one or more reservoirs (also referred to herein chamber(s)) prefilled with one or more fluid agents using a BFS manufacturing technique. In some embodiments, the reservoir module can have multiple reservoirs, and at least some of the reservoirs filled with fluid agents can be sealed from other of the reservoirs, thereby maintaining the fluid agents separate until combination thereof is desired (e.g., a time for use and/or administration to the patient). The reservoir module may be constructed, filled, and sealed, according to some embodiments, in a sterile manufacturing environment. BFS processes may, for example, offer a less expensive alternative to typical vials or bottles created via other manufacturing techniques. In some embodiments, the reservoir module (e.g., due to the nature of the BFS manufacturing process) may not require separate sterilization (e.g., and may accordingly be compatible with a wider array of fluid agents), may provide enhanced production rates of sterile/aseptic units per hour, and/or may be provided to an end-user for significantly lower per dose/unit costs. In some embodiments, these advantages may come with attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (November 15, 1989), which is incorporated herein by reference.

In some embodiments, a delivery module (also referred to herein as a manifold, coupling, or fluid sub-assembly) can be coupled to the reservoir module to breach one or more fluid seals thereof, thereby allowing fluid access to the one or more reservoirs (e.g., fluid agent can be added to and/or removed from the one or more reservoirs) of the reservoir module. In some embodiments, the reservoir module can have a neck that interfaces with the delivery module when the neck is inserted therein, which can facilitate coupling of the reservoir module to the delivery module. In some embodiments, the reservoir module includes a BFS vial having first and second reservoirs filled with respective first and second fluid agents. In some embodiments, first and second penetrable seals seal the first and second fluid agents within the first and second reservoirs. In some embodiments, the BFS vial includes a compressible main body containing the first and second reservoirs and a neck extending from the compressible main body. The neck includes first and second neck passages in fluid communication with the respective first and second reservoirs. The first and second seals are disposed at the end of the neck sealing the first and second neck passages.

In some embodiments, the delivery module includes a hub connector couplable to the BFS vial of the reservoir module and an administration member. The administration member pierces or penetrates the first and second penetrable seals upon coupling of the delivery module and the reservoir module to provide access to the first and second neck passages and to fluid agents within the first and second reservoirs. In embodiments, the administration member provides a multiple port ingress combining the first and second fluid agents within the administration member for delivery relative to the patient. In embodiments, the BFS vial of the reservoir module is actuated or compressed whereby the first and second fluid agents are dispensed from the first and second reservoirs, through the first and second neck passages and into the administration member.

In some embodiments, the administration member comprises a needle or cannula constructed for subcutaneous, intramuscular, intradermal, or intravenous injection of the combined fluid agents into the patient. Alternatively, in some embodiments, the administration member comprises a nozzle. For example, the nozzle can be a spray nozzle that facilitates dispersion of the combined liquid agents into a spray, which configuration may be useful in the administration of the combined fluid agents into a body cavity or orifice (e.g., nasal passage, ear canal, etc.). In another example, the nozzle can be a droplet nozzle that facilitates formation of droplets of the combined fluid agents, which configuration may be useful in the administration of the combined fluid agents to the eyes, for topical application, etc.

In some embodiments, the delivery module including the hub connector and the administration member may be configured to be coupled and/or assembled to the reservoir module on-site and/or in the field. Alternatively, or additionally, in some embodiments, a delivery module may be coupled and/or assembled to a reservoir module in a manufacturing facility and provided to a user as a single, pre-assembled fluid sub-assembly (e.g., with a neck of the reservoir module inserted into a recess of the connector, but without breach of the first and second penetrable seals of the reservoir module). In some embodiments, the pre-filled multi-fluid medical delivery system may, for example, be capable of delivering combined fluid agents in a controlled manner and without requiring specialized skill in assembling and/or administering delivery of such agents.

II. Pre-filled Multi-Fluid Medical Delivery Assemblies

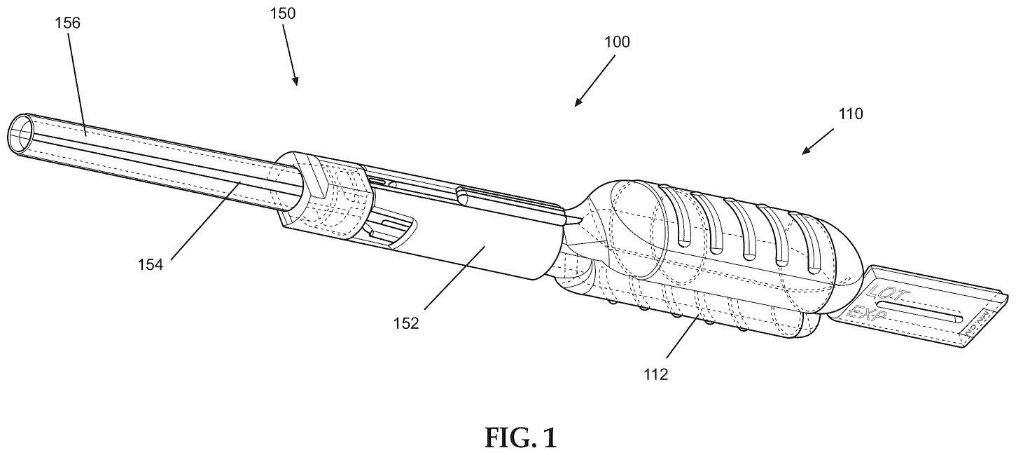

Referring initially to FIG. 1, a pre-filled multi-fluid medical system 100 according to some embodiments is illustrated. In some embodiments, the pre-filled multi-fluid medical system 100 may comprise various inter-connected and/or modular components, for example, a reservoir module 110 and a delivery module 150 which is couplable to the reservoir module 110. The reservoir module 110 includes multiple reservoirs filled with different fluid agents. In embodiments, the delivery module 150 is couplable to the reservoir module 110 at the procedural site, and includes the components to deliver the fluid agents to a patient.

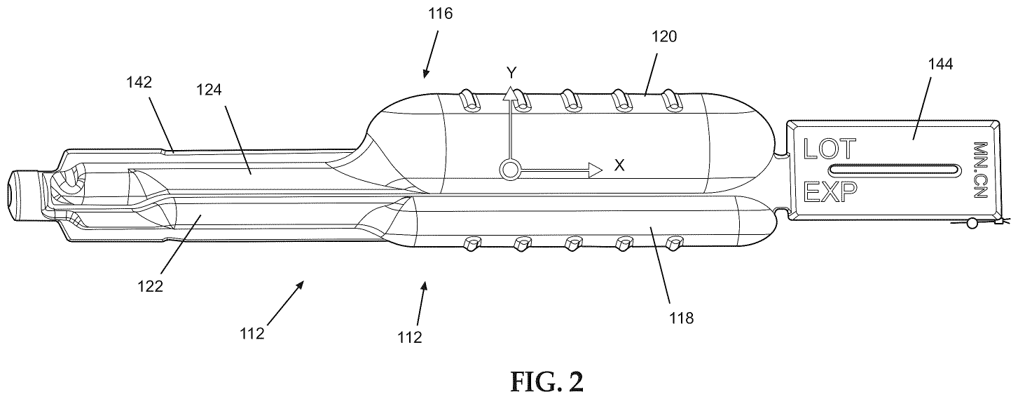

With reference to FIGS. 2-4, in conjunction with FIG. 1, the reservoir module 110 will be discussed. The reservoir module 110 includes a BFS vial 112 having a first vial component 114 and a second vial component 116 integrally formed with the first vial component 114. The vial 112 including the first vial component 114 and the second vial component 116 may be constructed (e.g., formed) via a BFS process and may comprise a “soft” plastic (e.g., having a Shore/Durometer “OO” hardness of between 60 and 70 and/or a Shore/Durometer “A” hardness between 20 and 50) that is not functionally susceptible to the formation and/or utilization of threaded connection features (as are possible to form on different, harder plastics). During the BFS manufacturing process, the vial 112 is formed, filled, and sealed in a continuous process without human intervention, in a sterile enclosed area inside a machine. In particular, the BFS manufacturing process involves multiple steps in which a pharmaceutical-grade plastic resin is vertically heat extruded through a circular opening to form a hanging tube called a parison. This extruded tube is then enclosed within a two-part mold, and the tube is cut above the mold. The mold is transferred to the filling zone, or sterile filling space, where filling needles (mandrels) are lowered and used to inflate the plastic to form the container within the mold. Following the formation of the vial 112, the mandrels are used to fill the vial 112 with one or more fluid agents. Following filling, the mandrels are retracted and a secondary top mold seals the container. All actions take place inside a sterile shrouded chamber inside the machine. The product is then discharged to a non-sterile area for labeling, packaging and distribution. In association with the BFS process, multiple vials 112 are produced and connected to each other as one or more trays or units comprising rows and columns of vials 112. Further details of compressible vials suitable for use with the medical tray apparatus 10 are illustrated in U.S. provisional patent Application Ser. No. 63/461,214, filed Apr. 21, 2023 and entitled “Syringe Cartridge System” and in international patent application publication number WO2017/187262 A1, published on 2 Nov. 2017 and entitled “Medical Delivery System,” the entire contents of each disclosure being incorporated by reference herein.

In embodiments, the first vial component 114 and the second vial component 116 are monolithically formed via the BFS process. More specifically, in embodiments, the first and second vial components 114, 116 are not separate elements but are formed as a single unit comprised as part of the vial 112. In some embodiments, the first and second vial components 114, 116 may be separate components (for example, separate BFS components) connected to each other via conventional methodologies. The first vial component 114 and the second vial component 116 include respective first and second compressible main bodies 118, 120 and first and second necks 122, 124 extending longitudinally from the first and second main bodies 118, 120. In embodiments, the first and second compressible main bodies 118, 120 collectively form the main body of the vial 112 and the first and second necks 122, 124 collectively form the neck of the vial 112. The vial 112 defines a longitudinal axis “x” and a transverse axis “y” orthogonal to the longitudinal axis ‘x”(FIG. 2).

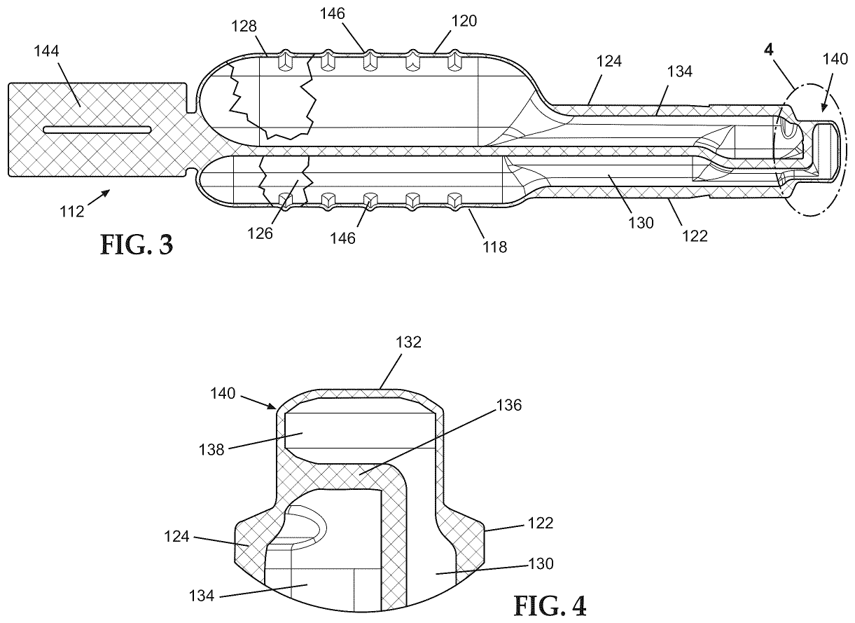

As best depicted in FIGS. 3 and 4, in embodiments, the first compressible main body 118 defines a first fluid reservoir 126 (shown in the cutaway of FIG. 3) of the first vial component 114 and the second compressible main body 120 defines a second fluid reservoir 128 (also shown in the cutaway of FIG. 3) of the second vial component 116. The first and second fluid reservoirs 126, 128 accommodate first and second fluid agents therein (not separately shown). According to some embodiments, the first and second fluid reservoirs 126, 128 are filled with a different fluid agent (e.g., having different composition, phase, volume, or any combination of the foregoing), such as a different liquid or gas. Such different fluids agents can be combined, introduced, and/or mixed via the pre-filled multi-fluid medical system 100 to define a combined fluid agent for use or administration to a patient. For example, the first fluid reservoir 126 can contain a first fluid agent, and the second fluid reservoir 128 can contain a second fluid agent. In some embodiments, the second fluid reservoir 128 may house or contain a second volume (e.g., two to three milliliters (2.0 to 3.0-ml)) of, for example, a vaccine or second agent (e.g., Pralidoxime). Alternatively or additionally, in some embodiments, the first fluid reservoir 126 may house or contain a first volume (e.g., seven tenths milliliters (0.7-ml) of, for example, an adjuvant (and/or carrier fluid, catalyst, diluent, etc.) or first agent (e.g., Atropine).

According to some embodiments, the first fluid reservoir 126 can have a first capacity (e.g., a volume for fluid agents), and/or the second fluid reservoir 128 can have a second capacity (e.g., a volume for fluid agents). In some embodiments, the second capacity can be greater than the first capacity, and/or the volume of the second fluid agent contained within the second fluid reservoir 128 can be greater than the volume of the first fluid agent contained within the first fluid reservoir 126. In some embodiments, the first and second fluid reservoir 126, 128 may define the same capacity. According to some embodiments, any or all of the fluid agents may be injected into the first and second fluid reservoirs 126, 128 of the vial 112 in a sterile environment during manufacture via a BFS process and sealed within the reservoir module 110.

The first neck 122 of the first vial component 114 defines a first neck passage 130 in fluid communication with the first fluid reservoir 126 and terminating in a first penetrable seal 132. The second neck 124 of the second vial component 116 defines a second neck passage 134 in fluid communication with the second fluid reservoir 128 and terminating in a second penetrable seal 136. In some embodiments, the first and second penetrable seals 132, 136 are in longitudinal alignment with each other. In embodiments, the first penetrable seal 132 is distal of the second penetrable seal 136 whereby a fluid chamber 138 is defined within the first neck passage 130 (e.g., between the first and second penetrable seals 132, 136) to enable mixing of the first and second fluid agents. In some embodiments, the first and second fluid agents are not mixed in any meaningful sense but may rather be delivered into the patient at essentially the same time or in sequence via the same needle. More than two necks are also contemplated to distribute three or more fluid agents.

In some embodiments, the first and second penetrable seals 132, 136 may comprise portions of the first and second vial components 114, 116, for example, that are configured to be breached (e.g., pierced) to expel the respective fluids. In some embodiments, the first penetrable seal 132, second penetrable seal 136, or both can be formed during a BFS manufacturing process. In some embodiments, the second penetrable seal 136 is thicker in cross-section or defines a thicker web resulting from fusing of materials during the BFS process. Alternatively, or additionally, in some embodiments, the first penetrable seal 132, second penetrable seal 136, or both may comprise foil, wax, paper, plastic, and/or other thin, pierceable objects or layers coupled to (or formed as part of) the vial 112. In some embodiments, one or both of the first penetrable seal 132 and the second penetrable seal 136 can provide a flat or planar piercing surface and/or can be oriented normal to an axis of the vial 112. In some embodiments, the first and second penetrable seals 132, 136 can be breached, for example, by puncturing, piercing, rupturing, penetrating, or otherwise breaking the seal, in order to access and/or dispense contents of the first and second fluid reservoirs 126, 128. In some embodiments, the first penetrable seal 132 and/or the second penetrable seal 136 defines a diameter ranging from about 1 millimeter (mm) to about 3 mm or about 1.5 mm.

In some embodiments, the neck of the vial 112 defines a distal connection fitting or interface 140 surrounding the fluid chamber 138. In embodiments, the connection interface 140 defines a reduced cross-section or diameter and cooperates with corresponding structure of the delivery module 150 to couple the reservoir module 110 and the delivery module. In some embodiments, the connection interface 140 is slightly deformable or compressible to be received within corresponding structure of the delivery module 150, and may expand outwardly once positioned within the delivery module 150 to facilitate and/or establish a coupling relation. In some embodiments, the connection interface 140 may have a surface feature, such as one or more protrusions, for retaining the connection interface 140 within a recess of the delivery module 150.

With reference again to FIG. 2, the vial 112 may include a pair of diametrically opposed wings 142 depending radially outwardly with respect to the longitudinal axis “x.” In some embodiments, the opposed wings 142 are arranged in diametric opposed relation. The wings 142 are narrow in cross-section defining a thickness, in some embodiments, less than one (1) millimeter. In embodiments, the wings 142 assist in aligning the vial 112 with respect to the delivery module 150 during coupling thereof and may also assist in retaining the vial 112 within the delivery module 150. The vial 112 may further include a vial handle 144. The vial handle 144 may be planar and include indicia or an indicator (such as an RFID chip) representative of the type, lot, expiration date of the fluid agent contained within the vial body 20. The wings 142 and the vial handle 144 may be formed during the BFS manufacturing process. The vial 112 may comprise protrusions 146 on the outer surfaces of the first and second compressible main bodies 118, 120 to assist in handling of the vial 112.

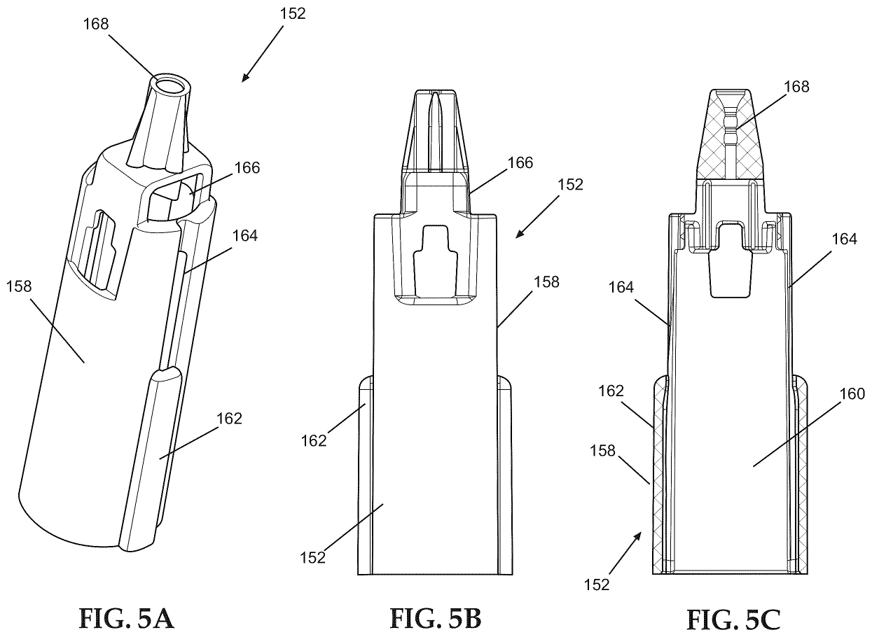

With reference again to FIG. 1, the delivery module 150 will be discussed. At least some features of the delivery module 150 are disclosed in commonly assigned PCT Application Serial No. PCT/US23/25123, filed Jun. 13, 2023, the entire contents of which are incorporated herein. The delivery module 150 includes a hub connector 152, an administration member 154 secured to the hub connector 152 and a protective cover 156 positionable over the administration member 154. The hub connector 152 is shown in FIGS. 5A-5C. The hub connector 152 includes a connector housing 158 defining an internal housing chamber 160 for reception of the neck and the connection interface 140 of the vial 112. The connector housing 158 further includes one or more alignment tracks 162 configured for reception of at least portions of the wings 142 of the vial 112. The alignment tracks 162 lead to open channel segments 164. The connector housing 158 further includes a pair of opposed windows 166 distal of the open channel segments 164. During coupling and insertion of the vial 112 to the hub connector 152, the connection interface 140 of the vial 112 is received within the internal housing chamber 160 with the wings 142 aligned to traverse the alignment tracks 162. The wings 142 are advanced within the alignment tracks 162 and are received within the open channel segments 164. Simultaneously therewith, the connection interface 140 advances within the internal housing chamber 160 and, in embodiments, is at least partially received within the opposed windows 166 of the connection housing 158. In some embodiments, the connection interface 140 may be compressed during passage through the internal housing chamber 160 and then assume a normal expanded state secured within the opposed windows 166. These features secure the delivery module 150 relative to the reservoir module 110. Alternatively or additionally, the connection interface 140 may comprise threads that correspond and cooperate with threads of the connector housing 158 such that they may be rotationally and/or removably coupled. Further details of the securement feature may be ascertained by reference to the PCT ‘123 application.

The connection housing 158 further includes a fluid outlet port 168 in fluid communication with the internal housing chamber 160 and the first and second fluid reservoirs 126, 128 of the vial 112 (for example, subsequent to penetration of the first and second penetrable seals 132, 136). In embodiments, the administration member 154 is secured within the fluid outlet port 168 via conventional methodologies including adhesives, cements or the like.

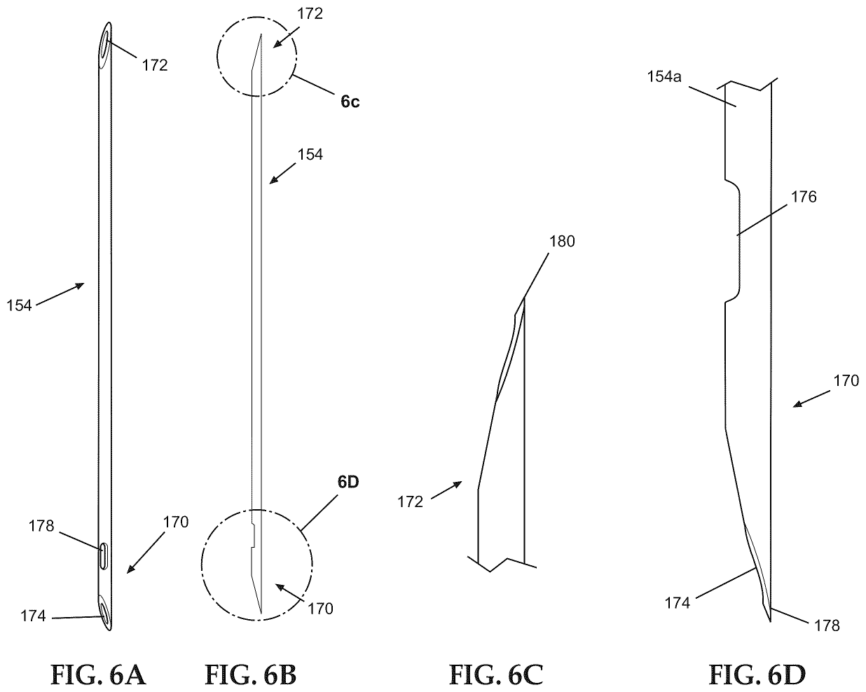

Referring now to FIGS. 6A-6D, the administration member 154 will be discussed. In some embodiments, the administration member 154 is a delivery member and, in embodiments, may include a needle for at least one of subcutaneous, intramuscular, intradermal, and intravenous injection of the fluid agent into the patient. However, it should be noted that, in other embodiments, the administration member 154 may include a nozzle (not shown) configured to control administration of the fluid agent to the patient. The nozzle may include a spray nozzle, for example, configured to facilitate dispersion of the fluid agent into a spray or a droplet nozzle configured to disperse the fluid agent in the form of one or more drops.

In some embodiments, the administration member 154 is a cannulated needle having one or more needle passages 154a and having a vial penetrating end 170 and an opposed patient end 172. The vial penetrating end 170 of the cannulated needle is disposed within the interior of the connector housing 158 at a predetermined longitudinal location to pierce the first and second penetrable seals 132, 136 of the vial 112 when the delivery module 150 is coupled to the reservoir module 110. In embodiments, the needle includes an axial port 174 in alignment with the passage 154a or lumen of the needle and a side port 176 spaced (for example, longitudinally spaced) spaced from the axial port 174. The axial port 174 and the side port 176 are in fluid communication with the needle passage 154a. In other embodiments, the axial port 174 may be a side port. The needle defines a penetrating tip 178 adjacent the axial port 174. The penetrating tip 178 may be beveled or pointed. As will be discussed in detail hereinbelow, when the delivery module 150 is coupled to the reservoir module 110, the axial port 174 is disposed within the second neck passage 134 of the vial 112 and the side port 176 is disposed within the fluid chamber 138 of the first neck passage 130 of the vial 112. In embodiments, the dimensions of the axial port 174 and the side port 176 may be selectively chosen to control the volume and/or rate of fluid flow of the fluid agents from the first and second neck passages 130, 134 into the needle passage 154a. This provides flexibility in controlling mixing and/or distribution of the fluid agents.

In embodiments, the patient end 172 of the needle may include a sharpened tip 180 configured for subcutaneous, intramuscular, intradermal, and intravenous injection of the fluid agent into the patient. In some embodiments, the needle may have multiple passages including a first passage in communication with the axial port 174 and a second passage in communication with the side port 176. In some embodiments, in which, for example, the administration member 154 is to be used as a dispensing nozzle, the patient end 172 may be replaced with an atraumatic feature such as a nozzle to avoid penetration of the subject. In some embodiments, the administration member 154 in the form of a needle may be constructed of a metal or a hard plastic material.

In some embodiments, the administration member 154 may comprise a needle for at least one of subcutaneous, intramuscular, intradermal, and intravenous injection of the fluid agent into the patient. For example, the needle can have a length of 20 to 70 mm, inclusive, or about 50 mm and a diameter of 0.5 mm to 1.5 mm or about .8 mm. Other dimensions are contemplated.

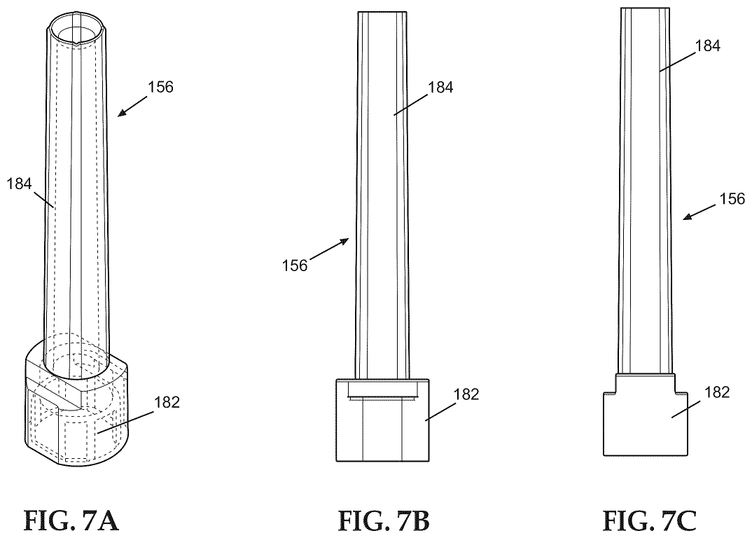

FIGS. 7A-7C illustrate details of the protective cover 156. The protective cover 156 includes a cover housing 182 and an elongated cover component 184 depending from the cover housing 182. The cover housing 182 includes inner wall portions defining an internal cover chamber configured to fit over the connector housing 158 whereby, for example, an interference with the exterior of the connector housing 158 is established between the components. The elongated cover component 184 encloses the administration member 154 during transport and prior to use. In some embodiments, the protective cover 156 remains connected to the connector housing 158 during coupling of the reservoir module 110 and the delivery module 150.

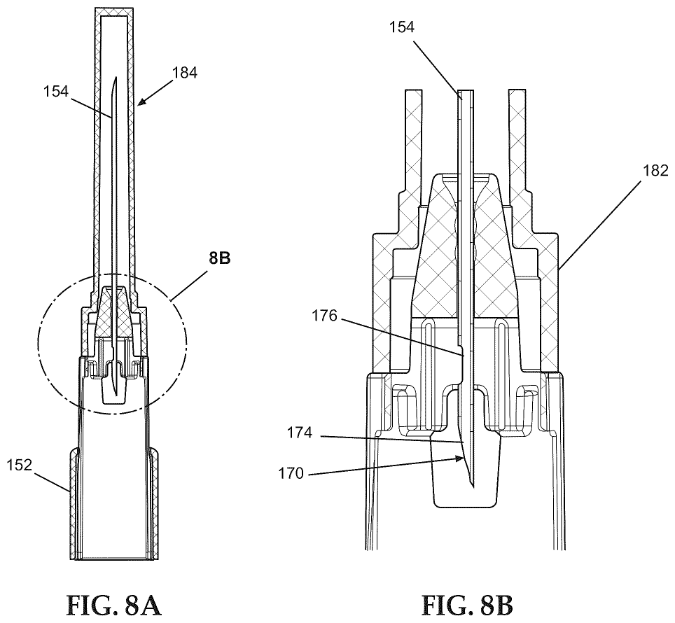

FIG. 8A illustrates, in a side cross-sectional view, the delivery module 150 in an assembled condition. FIG. 8B is an enlarged isolated view of the area of detail identified in FIG. 8A. As depicted, in the assembled condition the vial penetrating end 170 of the administration member 154 extends for a predetermined distance within the internal housing chamber 160 of the hub connector 152 sufficient to penetrate the first and second penetrable seals 132, 136 of the vial 112 when the delivery module 150 is coupled to the reservoir module 110.

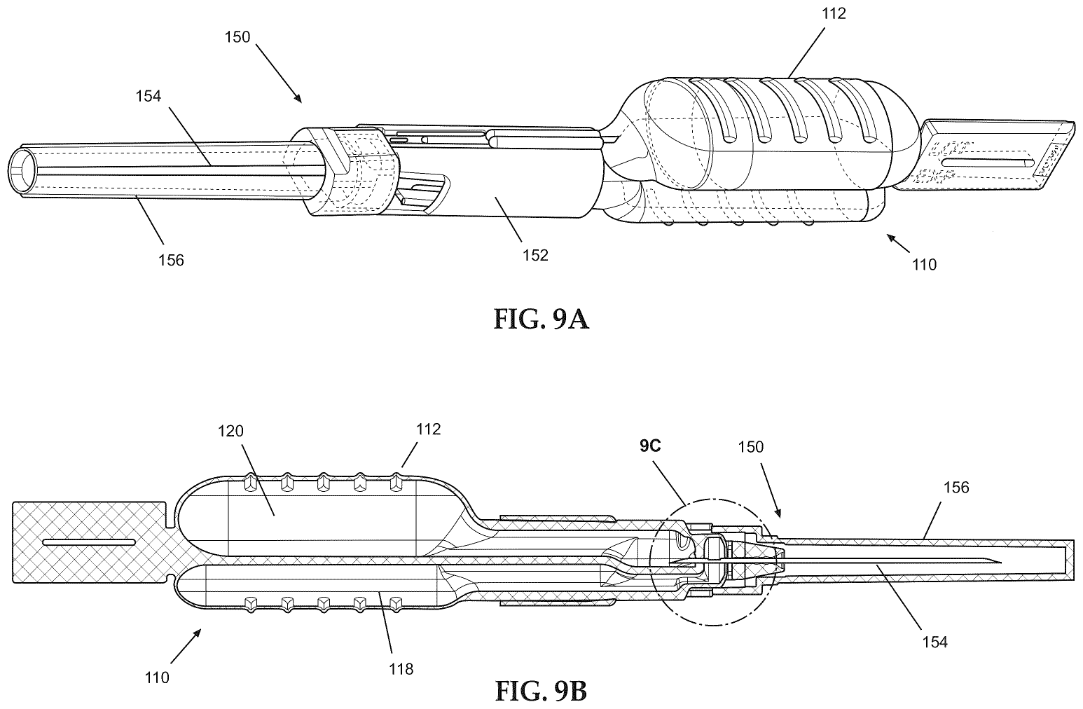

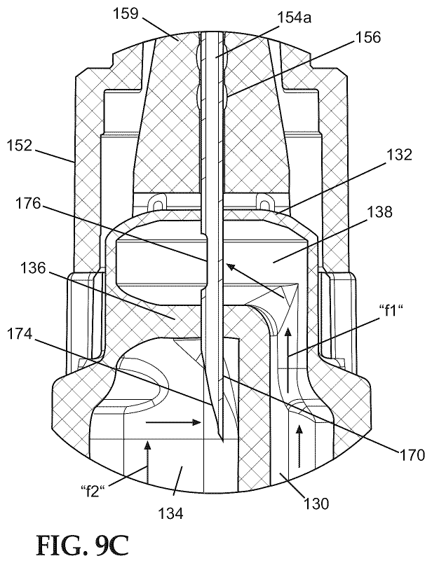

FIGS. 9A-9C illustrate the delivery module 150 coupled to the reservoir module 110. During the coupling sequence, the neck of the vial 112 is longitudinally aligned with the internal housing chamber 160 of the hub connector 152 with the wings 142 of the vial 112 also aligned with the alignment tracks 162 of the hub connector 152. The neck of the vial 112 is advanced within the internal housing chamber 160 of the hub connector 152. In embodiments, the connection interface 140 of the vial 112 is compressed during passage through the internal housing chamber 160 until it reaches the opposed windows 166 where it expands outwardly to be secured against/within the surfaces or ledges defining the windows 166. In embodiments, the wings 142 of the vial 112 may be compressed within the alignment tracks 162 during advancing movement of the vial 112, and also expand outwardly upon reaching the open channel segments 164 of the connector housing 158, and retained by trailing edges of the connector housing 158 defining the open channel segments 164. In other embodiments, the vial 112 may be secured within the hub connector 152 by other means including threads, a friction or tolerance fit or the like.

During coupling of the delivery module 150 and the reservoir module 110, the vial penetrating end 170 of the needle advances to penetrate the first penetrable seal 132 and the second penetrable seal 136 to assume the position depicted in FIG. 9C. During this movement, the needle is stabilized relative to the vial 112 via the coupling arrangement provided by, in embodiments, at least the connection interface 140 with the hub connector 152 and the wings 142 with the alignment tracks 162. Thus, the vial penetrating end 170 is retained in axial alignment with the first and second penetrable seals 132, 136 of the vial 112 to ensure a clean piercing action therethrough without deviation of the vial penetrating end 170. For example, the vial penetrating end 170 is retained in coaxial arrangement with respect to the first and second penetrable seals 132, 136 to ensure direct penetration therethrough. In embodiments, the diameter of the needle is one-half the dimension of the diameter(s) of the first and second penetrable seals 132, 136 to accommodate, for example, slight off center deviation of the penetrating end 170. In this coupled position, the axial port 174 of the needle is disposed within the second neck passage 134 and the side port 176 of the needle is disposed within the fluid chamber 138 of the first neck passage 130.

With the needle accessing the vial 112 as shown, in response to, for example, a squeezing action on the first and second compressible main bodies 118, 120, the first fluid agent (represented by arrows “f1″) passes from the first fluid reservoir 126 through the first neck passage 130 and the fluid chamber 138 to be received within the side port 176 of the needle and the second fluid (represented by arrows “f2”) passes from the second fluid reservoir 128 through the second neck passage 134 and into the axial port 174 of the needle. The axial and side ports 174, 176 are, in embodiments, in fluid communication with the single needle passage 154a. The fluid agents “f1”, “f2” pass through the needle passage 154a for administration to the patient. In embodiments, during passage, the fluid agents “f1”, “f2” may be distributed to mix within the fluid chamber 138 of the first neck passage 130. In some embodiments, fluid agents “f1”, “”f2” pass through multiple needle passages 154a of the needle.

In some embodiments, the first and second compressible main bodies 118, 120 may be selectively squeezed, for example, in sequence or alternatively, to selectively deliver the first and second fluid agents “f1” “f2” to the patient.

In some embodiments, when the administration member 154 is in the form of a needle, the patient end 172 may be positioned to penetrate the cutaneous/subcutaneous tissue to permit entry of the first and second fluid agents “f1”, “f2” into the patient. In some embodiments, the protective cover 156 may be removed before or after coupling of the reservoir module 110 and the delivery module 150.

In some embodiments, once sufficient dispensing of the first and second fluid agents “f1”, “f2” from the vial 112 has been achieved (e.g., once the first and second fluid reservoirs 126, 128 have been emptied), the medical system 100 may be discarded. In some embodiments, the reservoir module 110 may be separated from the delivery module 150, and one or more of the modules discarded.

In some embodiments, the medical system 100 including the reservoir module 110 and the delivery module 150 can include additional components beyond those specifically illustrated hereinabove. In some embodiments, the components of the medical system 100 form a kit including components of the reservoir module 110 and the delivery module 150. Thus, the delivery module 150 and components thereof may not need to be stored or shipped in accordance with often restrictive requirements imposed on medicaments and may accordingly reduce the amount of space required for such specialized storage and/or shipping. The delivery module 150 may also or alternatively be manufactured, stored, and/or shipped in advance (e.g., at a first time) while the reservoir module 110 including the BFS vials 112 that are pre-filled with the fluid agents may be manufactured, stored, and/or shipped at a later time (e.g., a second time). In some embodiments, the delay between the first time and the second time may be lengthy without causing detrimental effects, as the delivery module may be stored, in some embodiments, indefinitely. In such a manner, the delivery module may be provided to be on-hand in advance of the availability and/or arrival of multiple reservoir modules, reducing supply chain constraints in the case of proactive mixing assembly procurement.

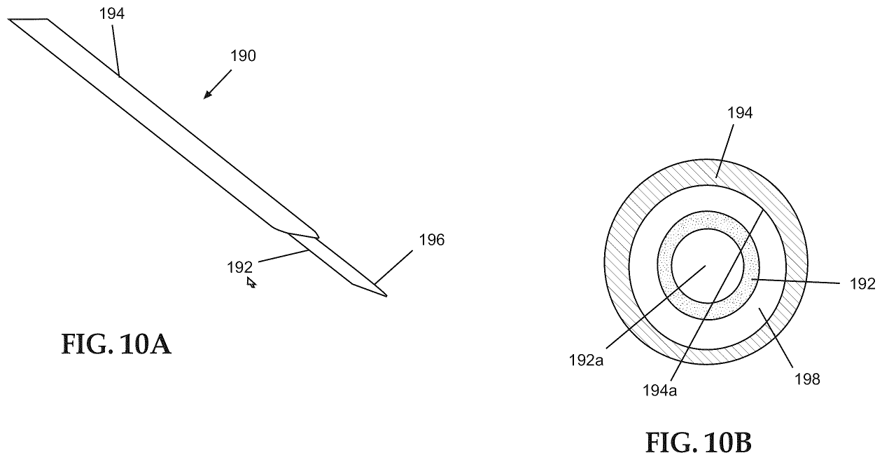

FIGS. 10A and 10B illustrate an administration member in the form a needle 190 including a first inner needle 192 and a second outer needle 194 disposed about the first inner needle 192, for example, in coaxial arrangement. The first inner needle 192 extends beyond the second outer needle 194 and has a penetrating end 196. The first inner needle 192 includes a needle lumen or passage 192a extending therethrough. The second outer needle 194 defines a lumen 194a which receives the first inner needle 192. The first inner needle 192 and the second outer needle 194 define a second needle passage 198 therebetween, for example between the outer surface of the first inner needle 192 and the inner surface of the second outer needle 194. The second needle passage 198 may be annular or partial annular in configuration. When coupled to the reservoir module, the penetrating end 196 of the first inner needle 192 is disposed within the second neck passage 134 and the end of the second outer needle 194 is disposed within the through the fluid chamber 138 of the first neck passage 130. (see FIG. 9C) The first fluid agent “f1” flows through the second needle passage 198 (defined between the first inner and second outer needles 192, 194) and the second fluid agent “f2” flows though the needle passage 192a of the first inner needle 192.

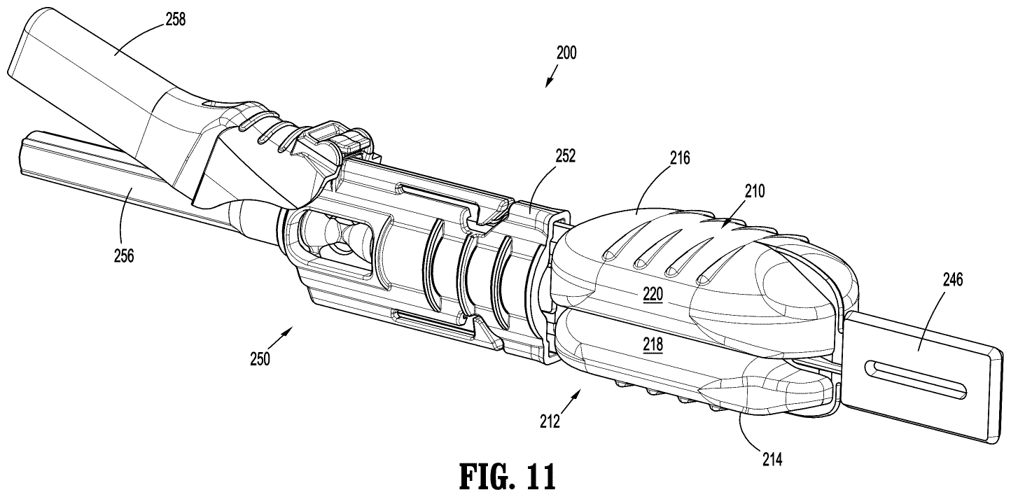

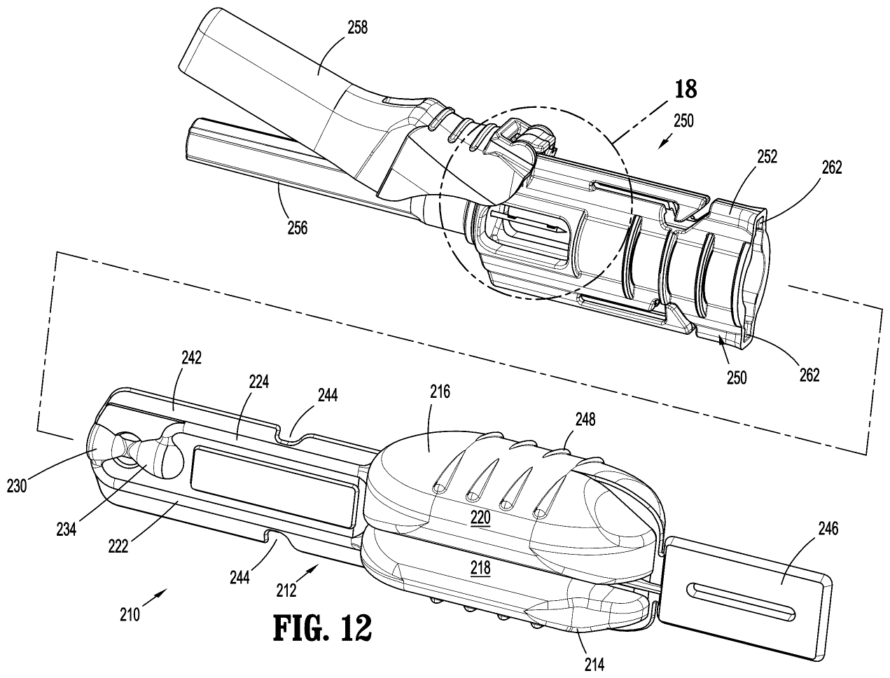

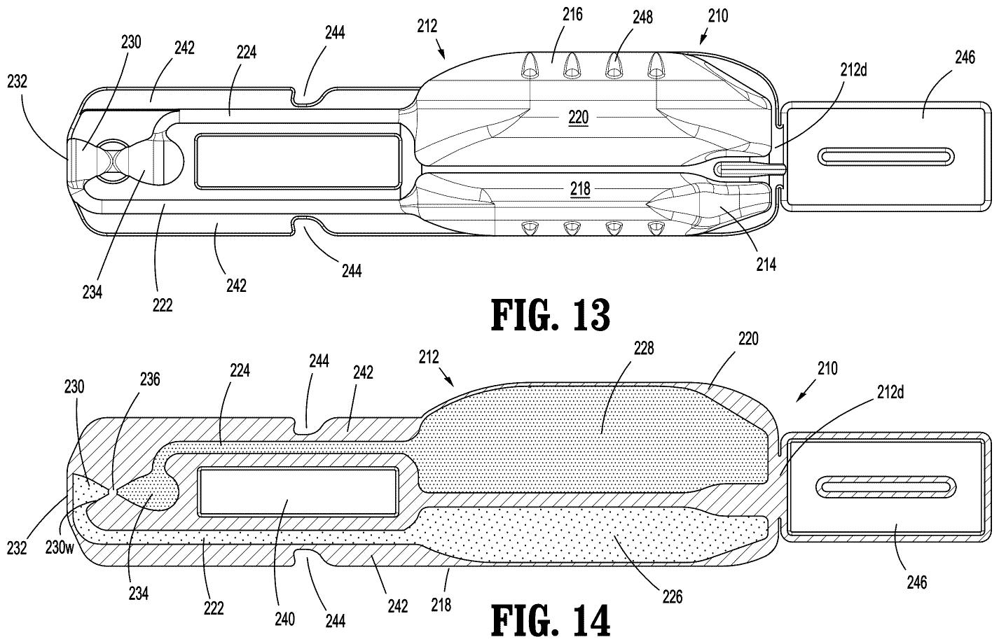

With reference to FIGS. 11-12, a pre-filled multi-fluid medical delivery system 200 according to one or more embodiments is illustrated. The pre-filled delivery system 200 includes a BFS vial or reservoir module 210 and a delivery module 250 couplable to the reservoir module 210. As best depicted in FIGS. 13-14 in conjunction with FIGS. 11-12, the reservoir module 210 includes a BFS vial 212 having a first vial component 214 and a second vial component 216 integrally formed with the first vial component 214. In embodiments, the first vial component 214 and the second vial component 216 are monolithically formed via the BFS process. The first vial component 214 and the second vial component 216 include respective first and second compressible main bodies 218, 220 and first and second neck passages 222, 224 extending longitudinally from the first and second main bodies 218, 220. In embodiments, the first and second compressible main bodies 218, 220 collectively form the main body of the BFS vial 212. The first and second neck passages 222, 224 collectively form at least part of the neck of the BFS vial 212.

As best depicted in FIGS. 12-14, the first compressible main body 218 defines a first fluid reservoir 226 of the first vial component 214 and the second compressible main body 220 defines a second fluid reservoir 228 of the second vial component 216. The first and second fluid reservoirs 226, 228 accommodate respective first and second fluid agents “f1”, “f2” therein. According to some embodiments, the first and second fluid reservoirs 226, 228 are filled with a different fluid agent (e.g., having different composition, phase, volume, or any combination of the foregoing), such as a different liquid or gas as described hereinabove.

According to some embodiments, the first fluid reservoir 226 can have a first capacity (e.g., a volume for fluid agents), and/or the second fluid reservoir 228 can have a second capacity (e.g., a volume for fluid agents). In some embodiments, the second capacity can be greater than the first capacity, and/or the volume of the second fluid agent “f2” contained within the second fluid reservoir 228 can be greater than the volume of the first fluid agent “f1” contained within the first fluid reservoir 226, for example, up to twice (2) the volume. In some embodiments, the first and second fluid reservoirs 226, 228 may define the same capacity.

The first neck passage 222 of the first vial component 214 is in fluid communication with the first fluid reservoir 226 and terminates in a first fluid chamber 230 sealed by a first penetrable seal 232. The second neck passage 224 of the second vial component 216 is in fluid communication with the second fluid reservoir 228 and terminates in a second fluid chamber 234 sealed by a second penetrable seal 236. In some embodiments, the first and second penetrable seals 232, 236 are in longitudinal alignment with each other. In embodiments, the first penetrable seal 232 is distal of the second penetrable seal 236 and is in spaced relation to the second penetrable seal 236. The distance between the first and second penetrable seals 232, 236 may range from about 2 mm to about 20 mm. In some embodiments, the first fluid chamber 230 is tapered, V-shaped or funnel-shaped which serves to guide the administration member during penetration at least through the second penetrable seal 236. In embodiments, the walls 230w defining the tapered first fluid chamber 230 assist in directing the needle toward the second penetrable seal 236.

In some embodiments, the second penetrable seal 236 is thicker in cross-section or defines a thicker web resulting from fusing of materials during the BFS process. In some embodiments, one or both of the first penetrable seal 232 and the second penetrable seal 236 can provide a flat or planar piercing surface and/or can be oriented normal to an axis of the BFS vial 212. In some embodiments, the first and second penetrable seals 232, 236 may include a contoured, or inwardly tapered surface to assist in guiding/aligning the needle. In some embodiments, the first and second penetrable seals 232, 236 can be breached, for example, by puncturing, piercing, rupturing, penetrating, or otherwise breaking the seal, in order to access and/or dispense contents of the first and second fluid reservoirs 226, 228. In some embodiments, the first penetrable seal 232 and/or the second penetrable seal 236 defines a diameter ranging from about 1 millimeter (mm) to about 3 mm or about 1.5 mm. In some embodiments, the BFS vial 212 may further include a depressed or thinned segment 240 between the first and second neck passages 222, 224. The thinned segment 240 may be formed during the BFS molding process, and may be a result of material transfer of the BFS plastic material toward the first and second neck passages 222, 224 to maintain the integrity and separation of the separate chambers.

The BFS vial 212 may include a pair of diametrically opposed wings 242 depending radially outwardly with respect to the longitudinal axis “x.” In some embodiments, the opposed wings 242 are arranged in diametric opposed relation. The wings 242 are narrow in cross-section defining a thickness, in some embodiments, less than one (1) millimeter. In embodiments, the wings 242 assist in aligning the BFS vial 212 with respect to the delivery module 250 during coupling thereof and may also assist in retaining the BFS vial 212 within the delivery module 250. The wings 242 further include locking recesses 244. The locking recesses 244 assist in securing the BFS vial 212 relative to the delivery module 250. The BFS vial 212 may further include a vial handle 246 and protrusions 248 on its outer surface. The BFS vial 212 may include an enlarged divider segment 212d adjacent the vial handle 246. The enlarged divider segment 212d may assist in maintaining the integrity of the BFS vial 212 while removing the BFS vial 212 from a strip of BFS vials and/or during manipulating of the BFS vial 212 relative to the delivery module 250. In some embodiments, the divider segment 212t may resist any tendency of tearing and/or leaking of the BFS vial 212 during use. The enlarged divider segment 212d may be formed during the BFS molding process.

The BFS vial 212 may include transparent sections or elongated transparent veins to enable the user to view the statuses of the first and second fluid agents “f1”, “f2” contained within the BFS vial 212 and/or confirm complete distribution of the first and second fluid agents “f1”, “f2” from the BFS vial 212.

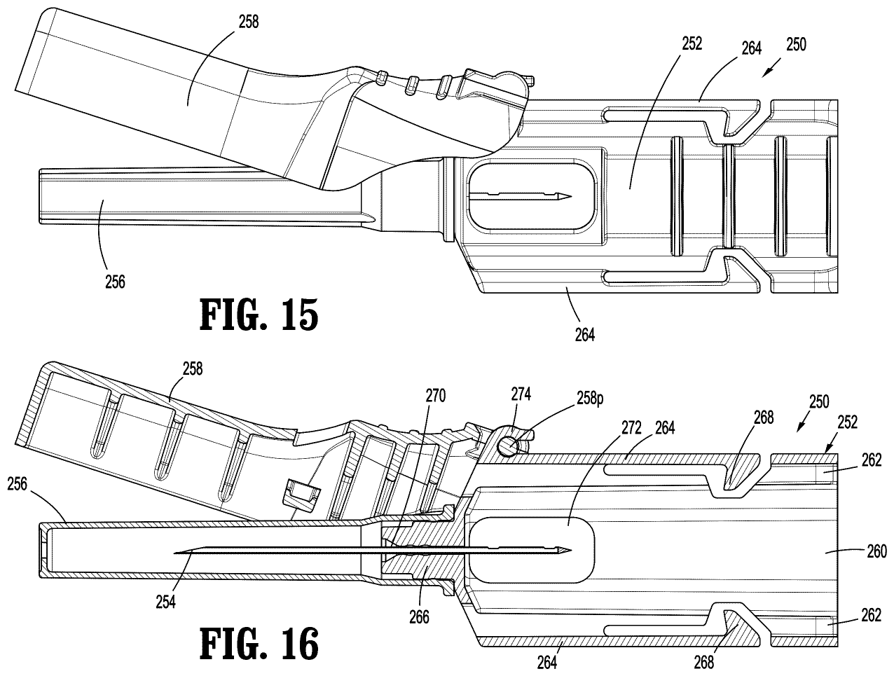

With reference to FIGS. 15-17, in conjunction with FIGS. 11 and 12, the delivery module 250 will be discussed. The delivery module 250 includes a connector body 252, an administration member 254 secured to the connector body 252, a protective cover 256 positionable over the administration member 254, and a pivoting shield 258 pivotally mounted to the connector body 252. The connector body 252 defines an internal housing chamber 260 for reception of the neck of the BFS vial 212. The connector body 252 further includes one or more alignment tracks 262 (FIG. 12) configured for reception of at least portions of the wings 242 of the BFS vial 212. The connector body 252 further includes a pair of a pair of locking arms 264 mounted to the connector body 252 and an administration hub connector 266. The locking arms 264 include locking detents 268 which selectively engage the locking recesses 244 of the BFS vial 212 to secure the delivery module 250 relative to the BFS vial 212. In embodiments, the locking arms 264 are normally biased such that the locking detents 268 are received within the locking recesses 244 of the BFS vial 212. The locking arms 264 exhibit spring-like characteristics to flex outwardly during mounting of the delivery module 250 on the BFS vial 212 and then return whereby the locking detents 268 are received within the locking recesses 244 thereby securing the delivery module 250 on the BFS vial 212. The administration hub connector 266 defines an opening 270 for reception and passage of the administration member 254. The administration member 254 is secured within the opening 270 of the administration hub connector 266 with adhesives, cements or the like. The connector body 252 may further include a pair of opposed windows 272 to enable viewing within the interior of the connector body 252 and the BFS vial 212.

The connector body 252 further includes a recessed mount 274 for mounting the pivoting shield 258 to enable pivotal movement of the pivoting shield 258. In embodiments, the recessed mount 274 is dimensioned to receive a corresponding dimensioned pivot pin 258p of the pivoting shield 258 to mount the pivoting shield 258 in pivotal relation to the delivery module 250.

The protective cover 256 includes a cover housing 278 and an elongated cover component 280 depending from the cover housing 276. The cover housing 276 includes inner wall portions defining an internal cover chamber configured to fit over the administration hub connector 266 whereby, for example, an interference fit with the exterior of the administration hub connector 266 is established between the components. The elongated cover component 280 encloses the administration member 254 during transport and prior to use. In some embodiments, the protective cover 256 remains connected to the administration hub connector 266 during coupling of the delivery module 250 to the BFS vial 212.

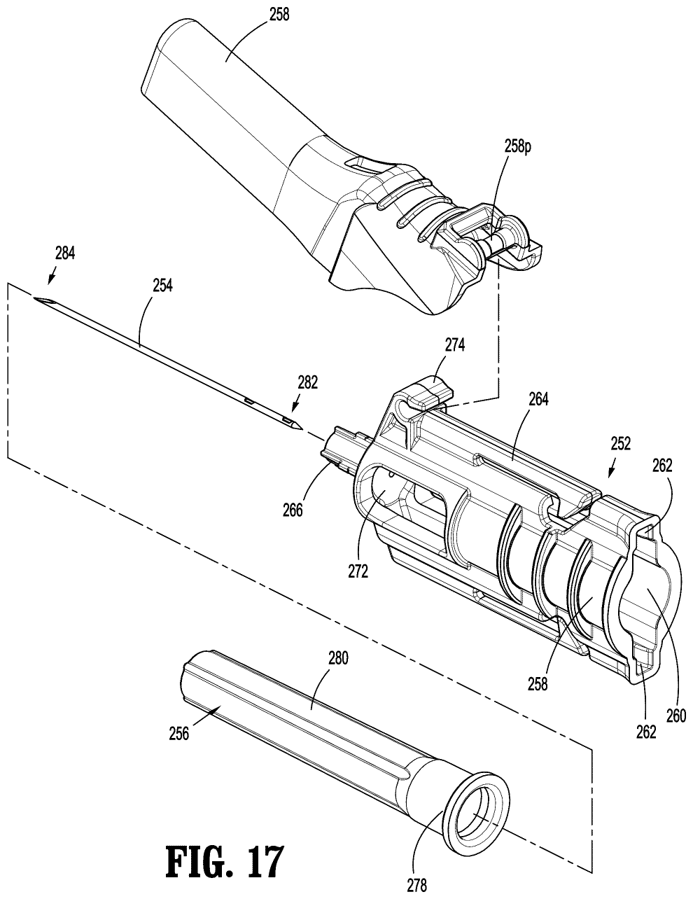

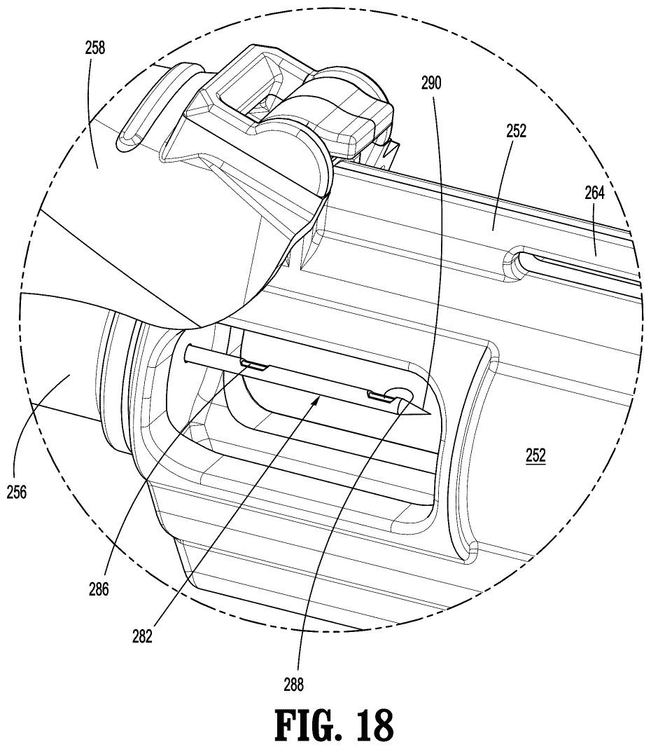

With reference to FIGS. 17 and 18, the administration member 254 may be a cannulated needle having one or more needle passages 254a and a vial penetrating end 282 and an opposed patient end 284. The cannulated needle may be a 20, 21, 22 or 23 gauge size needle. The vial penetrating end 282 is configured to pierce the first and second penetrable seals 232, 236 of the BFS vial 212 when the delivery module 250 is coupled to the reservoir module 210. In embodiments, the administration member 254 includes first and second side ports 286, 288 in fluid communication with the needle passage 254a or lumen of the needle. The side ports 286, 288 are longitudinally spaced. The needle defines a vial penetrating tip 290 which may be beveled or pointed. In embodiments, the penetrating tip 290 is a pencil-shaped tip. When the delivery module 250 is coupled to the reservoir module 210, the first side port 286 is disposed within the first fluid chamber 230 of the BFS vial 212 and the second side port 288 is disposed within the second fluid chamber 234 of the BFS vial 212. (FIG. 14). In embodiments, the dimensions of the side ports 286, 288 may be selectively chosen to control the volume and/or rate of fluid flow of the fluid agents from the first and second neck passages 222, 224 into the first and second fluid chambers 230, 234, and into the side ports 286, 288. For example, the side ports 286, 288 may be the same size or be different in size to offset the difference in volume of fluid agent “f1”, “f2” contained in the BFS vial 212, for example, and without limitation, to ensure that both liquids get injected at around the same rate or ratio (i.e., one doesn't get fully injected much before the other). This provides flexibility in controlling mixing and/or distribution of the fluid agents. In embodiments, the side port 286 may be smaller in size relative to the side port 286 due to potentially the smaller volume of fluid agent “f1” than the fluid agent “f2”. Other arrangement are also envisioned. The opposed patient end or administration end 284 of the cannulated needle is also tapered or pencil shaped in configuration.

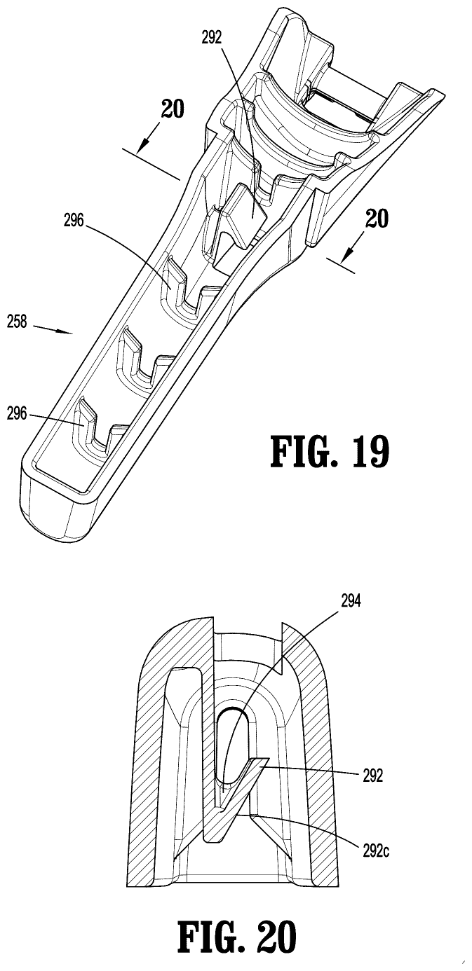

With reference to FIGS. 19 and 20, the pivoting shield 258 includes an internal locking latch 292. The internal locking latch 292 is configured to selectively secure the administration member 254 when the pivoting shield 258 is pivoted to cover the administration member 254. In embodiments, the internal locking latch 292 is sufficiently resilient to permit sliding passage of the locking latch 292 along outer cam surface 292c and then return under its own resiliency to capture the administration member 254 in locking recess 294 of the locking latch 292. In some embodiments, the locking latch 292 prevents reuse of the administration member 254, i.e., the pivoting shield 258 cannot be pivoted off the administration member 254 once secured to the administration member 254 without deforming and/or breaking the locking latch 292 and/or the administration member 254. In other embodiments, the locking latch 292 permits movement of the pivoting shield 258 off the administration member 254 subsequent to engagement with the administration member 254. The pivoting shield 258 includes internal support walls 296 with recesses for receiving the administration member 254 when the pivoting shield is mounted over the administration member 254.

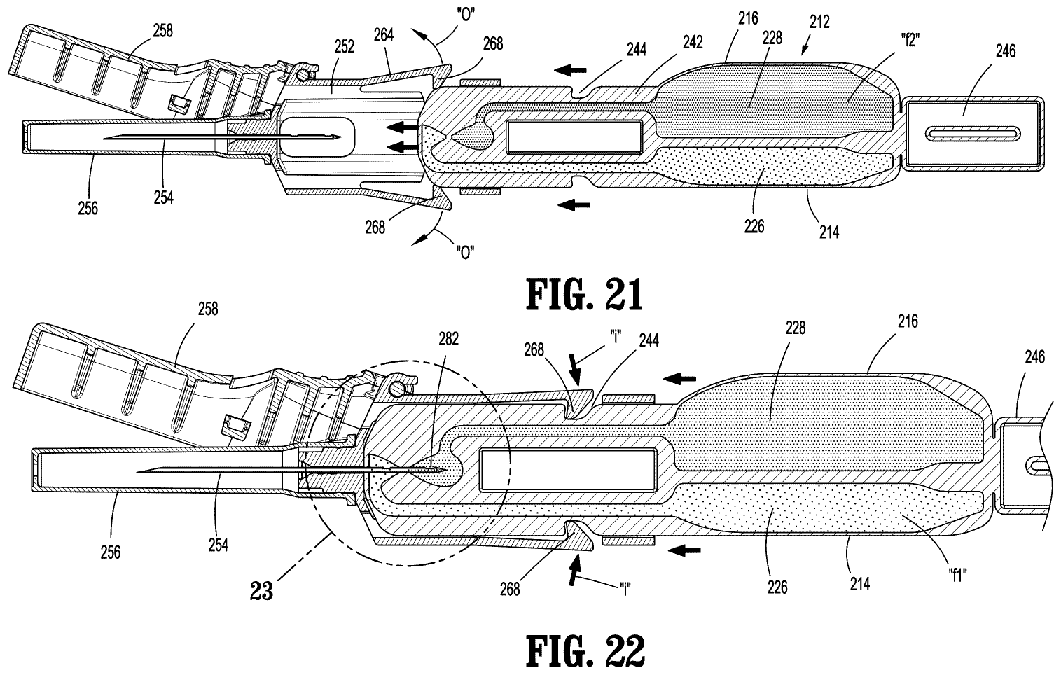

Referring now to FIGS. 21-23, the use of the delivery system 200 will be discussed. The BFS vial 212 of the reservoir module 210 is aligned with the delivery module 250 having the administration member 254, the protective cover 256 and the pivoting shield 258 in the assembled condition. In embodiments, the BFS vial 212 may be distributed independently or concurrently with the delivery module 250 to the procedural site. The BFS vial 212 is positioned within the internal housing chamber 260 of the connector body 252 with the wings 242 aligned with the alignment tracks 262. Upon insertion of the BFS vial 212, the locking arms 264 are biased outwardly (show as directional arrow “o”) through engagement with the opposed wings 242 of the BFS vial 212 as depicted in FIG. 21. The wings 242 are advanced to traverse within the alignment tracks 262. The BFS vial 212 is advanced continuously or relative to the connector body 252 until the locking detents 268 of the locking arms 264 are received within the locking recesses 244 of the wings 242 to secure the BFS vial 212 relative to the delivery module 210. The locking arms 264 move in the direction of directional arrows “i” (FIG. 22) under the influence of their own resiliency. In embodiments, the locking detents 268 create a snapping noise or action when received within the locking recesses 244 of the wings 242 of the BFS vial 212 and/or provide a tactile indicator identifying to the user of the proper coupling. In embodiments, once secured within the locking recesses 244, the locking detents 268 cannot be released without destroying the BFS vial 212. In other embodiments, the locking detents 268 may be manipulated for release from the locking recesses.

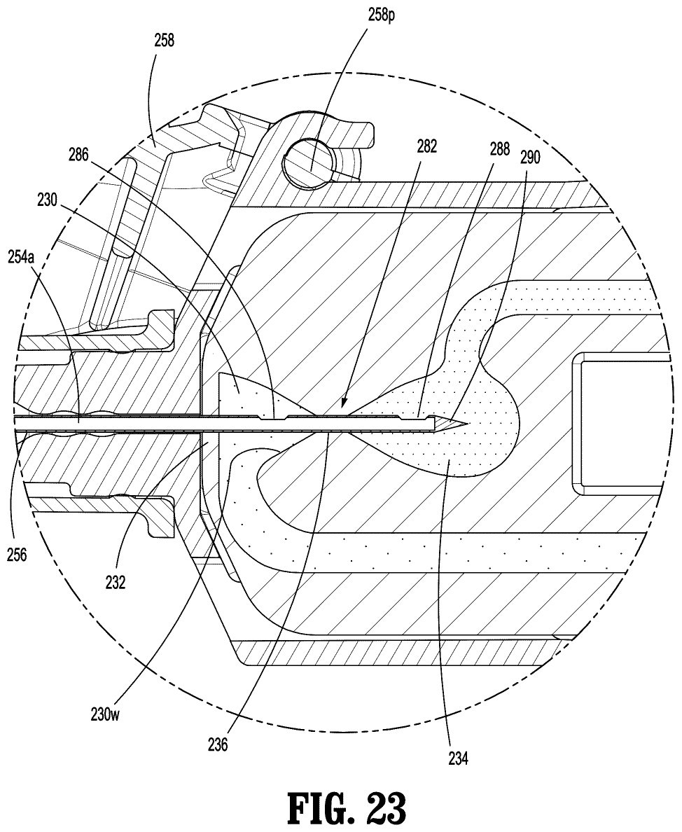

In conjunction therewith, during coupling of the delivery module 250 and the reservoir module 210, the vial penetrating end 282 of the administration member 254 advances to penetrate the first penetrable seal 232 and the second penetrable seal 236 to assume the position depicted in FIG. 23. During this movement, the administration member 254 is stabilized relative to the BFS vial 212 via the coupling arrangement provided by, in embodiments, at least by the wings 242 of the BFS vial 212 with the alignment tracks 262 of the connector body 252. Thus, the vial penetrating end 282 is retained in axial alignment with the first and second penetrable seals 232, 236 of the BFS vial 212 to ensure a clean piercing action therethrough without deviation of the vial penetrating end 282 of the administration member 254. For example, the vial penetrating end 282 is retained in coaxial arrangement with respect to the first and second penetrable seals 232, 236 to ensure direct penetration therethrough. In embodiments, the penetration force required to penetrate through the first penetrable seal 232 is different than the penetrating force required to pierce the second penetrating seal 236. In some embodiments, the internal chamber walls 230w defining the V-shape of the first internal chamber 230 assist in guiding and/or the vial penetrating end 282 toward the second penetrable seal 236. In embodiments, the diameter of the needle is one-half the dimension of the diameter(s) of the first and second penetrable seals 232, 236 to accommodate, for example, slight off center deviation of the vial penetrating end 282. In this coupled position, the first side port 286 of the administration member 254 is disposed within the first fluid chamber 230 of the BFS vial 212 and the second side port 288 of the administration member 254 is disposed within the second fluid chamber 234 of the BFS vial 212. The first and second side ports 286, 288 are in fluid communication with the passage 254a of the administration member 254.

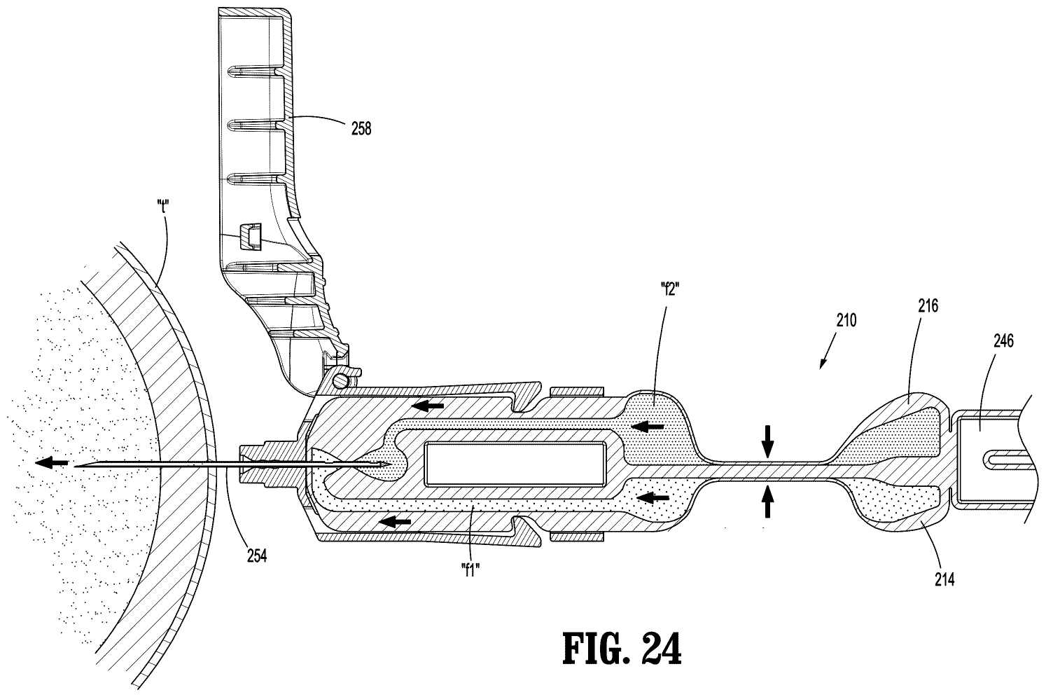

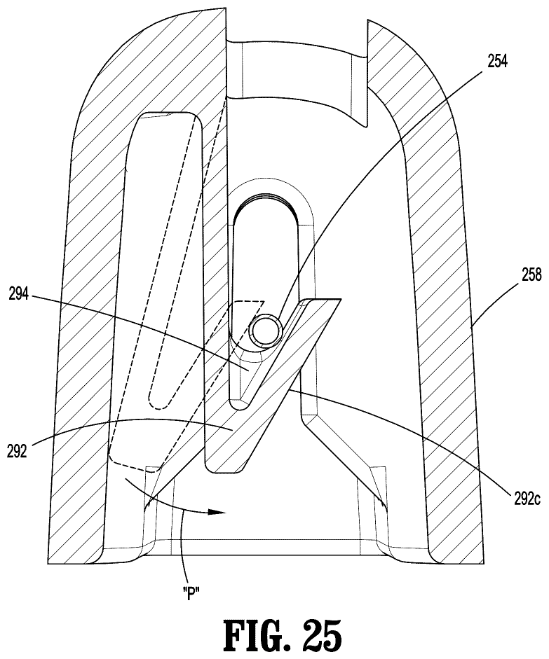

With the administration member 254 accessing the first and second fluid chambers 230, 234, the protective cover 256 is removed. As depicted in FIG. 24, the administration member 254 is introduced within tissue “t”. Thereafter, the user or clinician exerts a squeezing action the first and second vial components 214, 216 to cause the fluid agents “f1”, “f2” to be distributed through the first and second neck passages 222, 224 and into the respective first and second internal chambers 230, 234. The fluid agents “f1”, “f2” then enter the respective side ports 286, 288 of the administration for delivery through the passage 254a of the administration member 254 for delivery to the tissue “t” of the patient. When the fluid agents “f1”, “f2” are fully dispensed, the pivoting shield 258 may be selectively pivoted about pivot pin 258p in the direction of directional arrow “p” (FIG. 25) to cover the administration member 254. During positioning over the administration member 254, the cam surface 292c of the latch engages the administration member 254 to be biased outwardly and then returns under influence of its own resiliency whereby the administration member 254 is secured within the latch recess 294.

In some embodiments, the first and second vial components 214, 216 may be selectively squeezed, for example, in sequence, or alternatively, to selectively deliver the first and second fluid agents “f1” “f2” to the patient. In embodiments, the clinician can view the status of the fluid agents “f1”, “f2” through windows 272 to confirm if the fluids have been fully dispensed from the BFS vial.

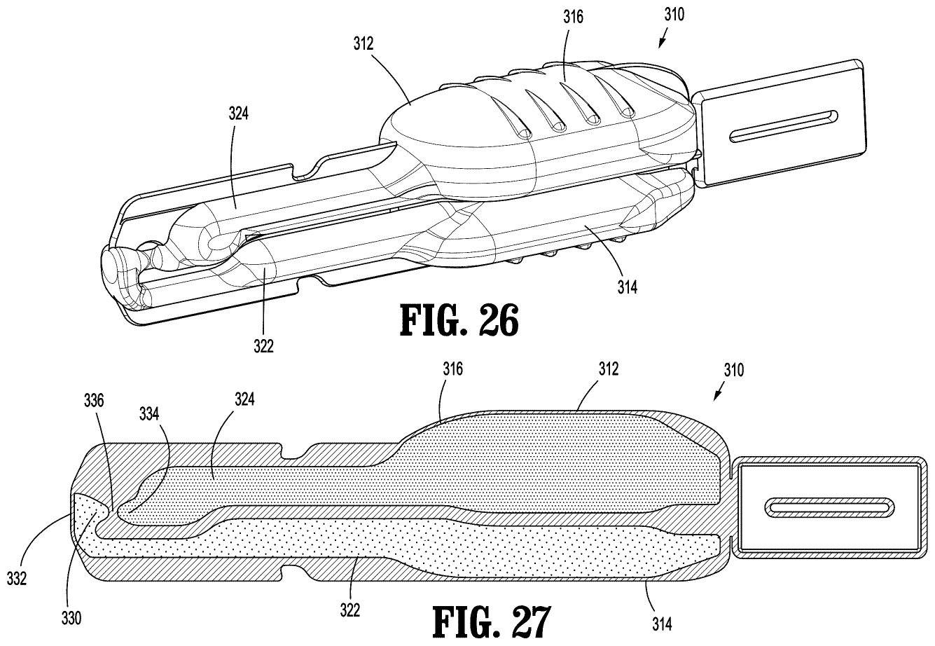

FIGS. 26 and 27 illustrate another reservoir module 310 for use with a pre-filled multi-fluid medical delivery system according to one or more embodiments of the present invention. The reservoir module 310 includes a BFS vial 312 having a first vial component 314 and a second vial component 316 with respective first and second compressible main bodies 318, 320 and first and second neck passages 322, 324. The first and second neck passages 322, 324 define a cross-sectional or internal dimension which is greater than corresponding neck passages of reservoir module 210. The first neck passage 322 terminates in a first fluid chamber 330 sealed by a first penetrable seal 332. The second neck passage 224 terminates in a second fluid chamber 334 sealed by a second penetrable seal 336. In embodiments, the first fluid chamber 330 is tapered or funnel-shaped which serves to guide the administration member during penetration at least through the second penetrable seal 336.

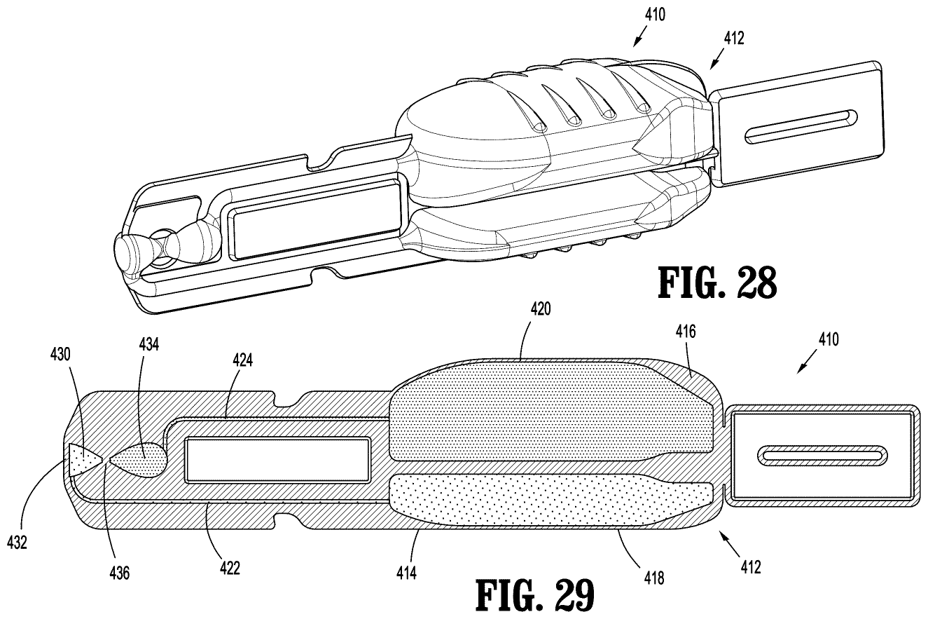

FIGS. 28-30 illustrate another reservoir module 410 for use with a pre-filled multi-fluid medical delivery system according to one or more embodiments of the present invention. The reservoir module 410 includes a BFS vial 412 having a first vial component 414 and a second vial component 416 with respective first and second compressible main bodies 418, 420 and first and second neck passages 422, 424. The first and second neck passages 422, 424 define a cross-sectional or internal dimension which is less than corresponding neck passages of reservoir modules 210, 310. The first neck passage 422 terminates in a first fluid chamber 430 sealed by a first penetrable seal 432. The second neck passage 424 terminates in a second fluid chamber 434 sealed by a second penetrable seal 436. In embodiments, the first fluid chamber 430 is tapered or funnel-shaped which serves to guide the administration member during penetration at least through the second penetrable seal 436. The length of the BFS vial 412 may be less than corresponding lengths of the BFS vials 212, 312.

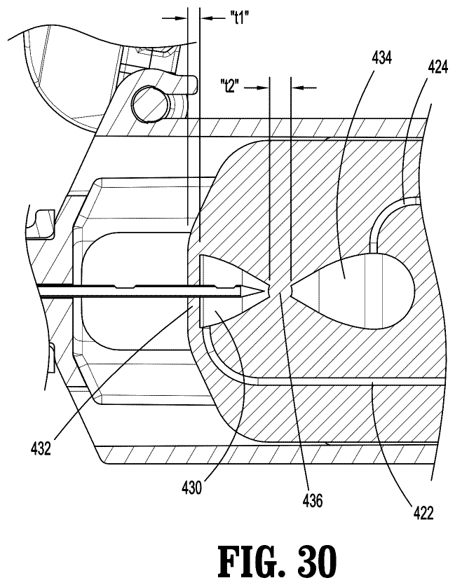

As best depicted in FIG. 30, in embodiments the second penetrable seal 436 defines a thickness “t2” greater than a corresponding thickness “t1” of the first penetrable seal 432. In embodiments, “t1” ranges from about 0.5 mm to about 1.5 mm and the thickness “t2” ranges from about 0.5 mm to about 2.5 mm. Other thicknesses are contemplated. Thus, in embodiments, the penetration force required for the administration member 254 to pass through the second penetrable seal 436 may be greater than the penetration force required to pass through the first penetrable seal 432. This may provide a tactile indicator to the clinician alerting the clinician of the location of the administration member 254 relative to the first and second penetrable seals 432, 436. In embodiments, the thicknesses “t1”, “t2” of the first and second penetrable seals 432, 436 may be selectively controlled to achieve defined penetration forces required for operation of the system. In other embodiments, the first penetrable seal 432 defines a thickness “t1” which is greater than the thickness “t2” of the second penetrable seal 436. For example, the thickness “t1” may be about 0.5 mm to about 0.7 mm and the thickness “t2” may be about 0.3 mm to about 0.5 mm. Thus greater force is required with the needle to penetrate the first penetrable seal 432 than the second penetrable seal 436.

FIG. 30 further illustrates the inner chamber walls 430w which assist in guiding the vial penetrating end 282 of the administration member 254 toward the second penetrable seal 436 during coupling of the components.

The BFS vial 412 includes one or more slits between the main body and the handle 446. The slits provide more rotational movement at the end of the BFS vial 412.

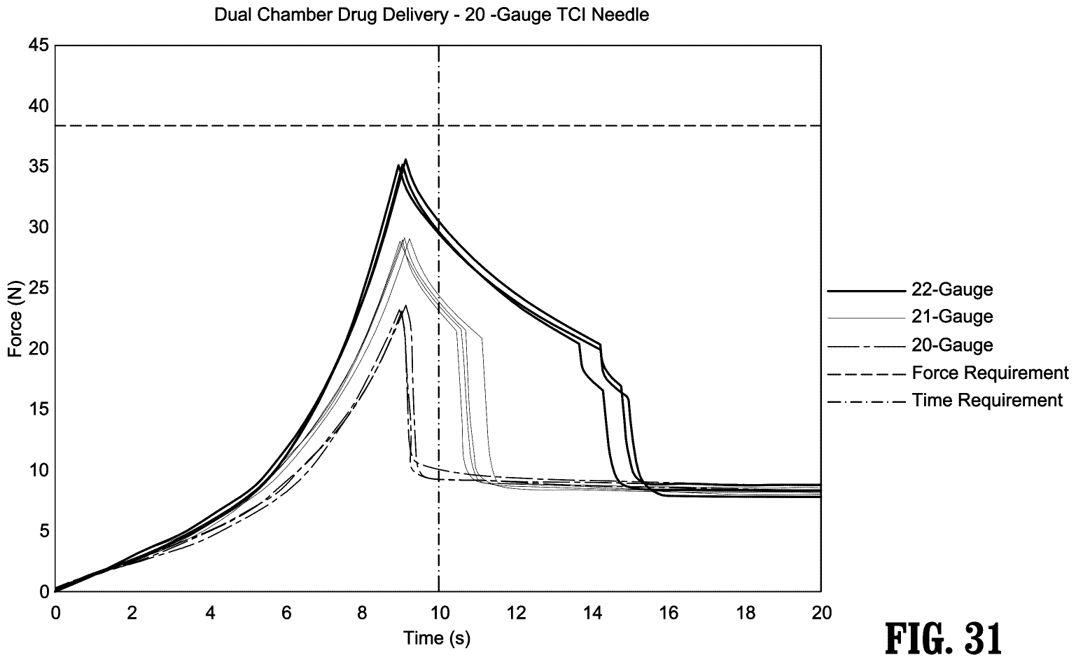

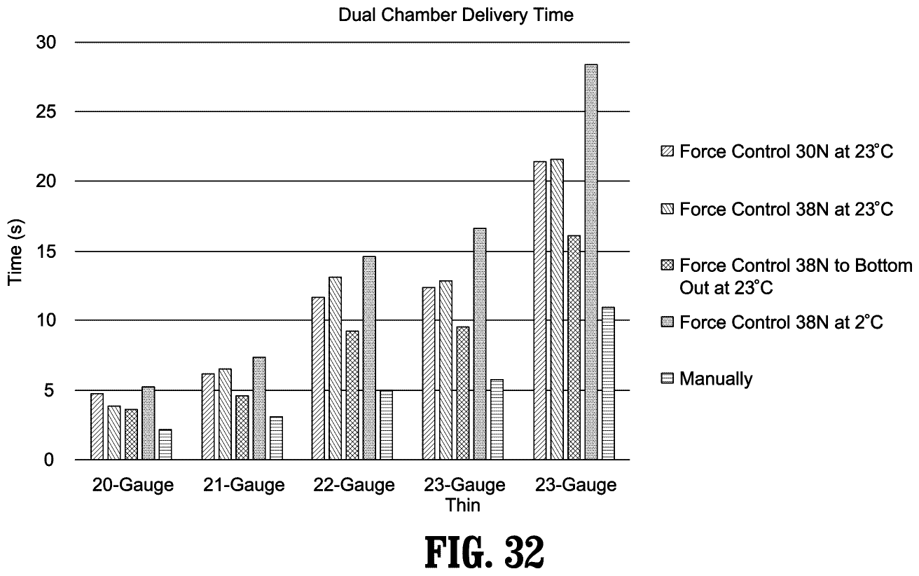

As described hereinabove, the delivery time and/or delivery forces for distribution of the first and second fluid agents “f1”, “f2” from the BFS vial 212, 312, 412 may vary based on agent type, temperature needle gauge size and/or size of the side ports 286, 288. In embodiments, decreasing the size of the needle will increase the delivery time and delivery forces (squeezing force on the vial 212) needed to expel and exhaust the fluid agents “f1”, “f2”. In general, the force required to deliver the fluid agents “f1”, “f2” from the BFS vial 212, 312, 412 may range from about 20 to about 35 Newtons for needle gauge sizes ranging from sizes 20, 21, 22, and 23 having nominal inner diameters ranging from 0.32 mm to about 0.55 mm. The amount of time required to exhaust the fluid agents “f1”, “f2” ranges from 10 to about 20 seconds. FIGS. 31 and 32 are graphs representing various gauge needles, delivery times and delivery forces for distribution of the fluid agents “f1”, “f2” from the vials 212, 312, 412. In embodiments, a 23 gauge needle may be used. The relatively small 23 gauge needle (opposed to larger 20-22 gauge needles) may minimize the potential for off-axis penetration of the needle end relative to the first and second penetrable seals 432, 436 (due in part to its minimal diameter) thus ensuring the needle ports 286, 288 are disposed within their respective chambers 230, 234 (FIG. 23) upon coupling of the components.

The present invention has application in other fields and technologies in addition to medical applications. The reservoir module and delivery module of the pre-filled delivery system may be used to deliver any multi-agent fluid combination in which the fluid agents are stored separately prior to mixing and use. For example, and without limitation, the pre-filled delivery system may be as a two component epoxy adhesive applicator. Other uses are also envisioned.

### III. Additional Examples of the Disclosed Technology

In view of the above described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

### IV. Rules of Interpretation

Any or all of the components disclosed herein can be formed of one or more plastics. In some embodiments, some components of the reservoir module (e.g., the BFS vials) can be formed of a relatively soft polymer (e.g., having a Shore/Durometer “D” hardness of between 60 and 70), such as polyethylene (e.g., low density polyethylene (LDPE), polypropylene, or any other polymer adaptable for use in a BFS manufacturing process. In some embodiments, some components of the delivery module (e.g., the connection assemblies, the administration assemblies, and/or needle caps or covers) can be formed, at least in part, of a relatively hard polymer (e.g., having a hardness greater than 80 on the Rockwell “R” scale), such as, but not limited to, polypropylene, polycarbonate, polybenzimidazole, acrylonitrile butadiene styrene (ABS), polystyrene, polyvinyl chloride, or the like. Other materials are also possible according to one or more contemplated embodiments.

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments.

Neither the Title (set forth at the beginning of the first page of this patent application) nor the Abstract (set forth at the end of this patent application) is to be taken as limiting in any way as the scope of the disclosed invention(s).

While the term “modules” is utilized herein for convenience and ease of illustration, objects represented and/or described as “modules” may comprise various forms, configurations, and/or quantities of components. A reservoir module may comprise one or more BFS vials and products that are formed and/or manufactured together or separately, for example, and/or may comprise one or more BFS chambers, bottles, containers, and/or other fluid-retaining objects. The term “module” does not convey any designation of shape or size. In some embodiments, a reservoir or BFS module may comprise one or more vials. According to some embodiments a reservoir module and/or a BFS vial may comprise one or more fluid reservoirs or chambers. In some embodiments, a plurality of BFS modules, components, vials, and/or chambers may be manufactured simultaneously from a single BFS mold. Each respective module and/or chamber may be formed, for example, by different portions of a single BFS mold (e.g., two cooperative halves thereof). In some embodiments, reservoir or BFS modules, components, vials, and/or chambers may be joined and/or coupled during manufacturing (e.g., via unformed and/or fused connecting parison) and/or after manufacturing/filling.

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget”does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality / features.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise.

Headings of sections provided in this patent application and the title of this patent application are for convenience only, and are not to be taken as limiting the disclosure in any way.

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A medical system, comprising:
a reservoir module defining a longitudinal axis, including:
a Blow-Fill-Seal (BFS) vial including a compressible body and a neck extending from the compressible body, the compressible body defining a first reservoir having a first fluid agent therein and a second reservoir having a second fluid agent therein;
a first penetrable seal within the neck for sealing the first fluid agent; and
a second penetrable seal within the neck for sealing the second fluid agent; and
a delivery module including an administration member configured to penetrate the first and second penetrable seals to access the first and second fluid agents for administration to a patient.

2. The medical system according to claim 1 wherein the neck defines a first neck passage in fluid communication with the first reservoir and terminating at the first penetrable seal and a second neck passage in fluid communication with the second reservoir and terminating at the second penetrable seal.

3. The medical system according to claim 2 wherein the first neck passage extends beyond the second neck passage.

4. The medical system according to claim 3 wherein the first penetrable seal is in general longitudinal alignment with the second penetrable seal.

5. The medical system according to claim 4 comprising a delivery module couplable to the reservoir module, including:
a hub for coupling to the vial of the reservoir module; and
the administration member coupled to the hub for distribution of the first fluid agent and the second fluid agent.

6. The medical system according to claim 5 wherein the administration member includes a needle, the needle having a vial penetrating end configured to penetrate the first and second penetrable seals of the vial upon coupling of the hub of the delivery module to the vial of the reservoir module and an administration end for delivering the first and second fluid agents to the patient.

7. The medical system according to claim 6 wherein the needle defines one or more needle passages for passage of the first fluid agent and the second fluid agent.

8. The medical system according to claim 7 wherein the needle defines a first needle port and a second needle port axially spaced from the first needle port, the first and second needle ports in fluid communication with the one or more needle passages of the needle.

9. The medical system according to claim 8 wherein, when the delivery module is coupled to the reservoir module, the first needle port is in fluid communication with the first neck passage and the first reservoir, and the second needle port is in fluid communication with the second neck passage and the second reservoir.

10. The medical system according to claim 9 wherein the vial of the reservoir module defines a fluid chamber in fluid communication with the first reservoir and with the second reservoir upon penetration of the vial penetrating end of the needle through the first penetrable seal and the second penetrable seal to enable mixing of the first fluid agent and the second fluid agent.

11. The medical system according to claim 7 wherein the needle includes a first inner needle and a second outer needle disposed about the first inner needle, the first inner needle defining a first needle passage, the first inner needle and the second outer needle defining a second needle passage therebetween.

12. The medical system according to claim 5 wherein the neck of the vial of the reservoir module includes a connection interface configured for at least partial reception within the hub of the delivery module.

13. A pre-filled medical delivery assembly comprising:
a reservoir module comprising a blow-fill-seal (BFS) vial including a first vial component having a first penetrable seal and a first reservoir with a first fluid agent therein and a second vial component having a second penetrable seal and a second reservoir with a second fluid agent therein, the BFS vial defining a longitudinal axis; and
a delivery module couplable to the BFS vial including:
a connector body for at least partially receiving the BFS vial; and
an administration member mounted to the connector body, the administration member including a vial end and a patient end, the vial end defining a first port and a second port longitudinally spaced relative to the first port;
wherein upon coupling of the BFS vial and the delivery module:
the vial end of the administration member penetrates the first penetrable seal and the second penetrable seal in succession whereby the first port of the administration member is in fluid communication with the first fluid reservoir of the first vial component and the second port of the administration member is in fluid communication with the second fluid reservoir of the second vial component to permit passage of the first and second fluid agents through the administration member.

14. The pre-filled delivery assembly of claim 13 wherein the first vial component defines a first chamber in fluid communication with the first fluid reservoir and the second vial component defines a second chamber in fluid communication with the first fluid reservoir, the first and second ports of the administration member being disposed within respective first and second chambers upon coupling of the BFS vial relative to the delivery module.

15. The pre-filled delivery assembly of claim 14 wherein the first and second ports of the administration member are side ports.

16. The pre-filled delivery assembly of claim 15 wherein the first side port has an opening defining a first internal dimension and the second side port has an opening defining a second internal dimension different form the first internal dimension.

17. The pre-filled delivery assembly of claim 13 wherein the connector body includes a pair of locking arms with locking detents, the locking detents being received within cooperating locking recesses of the BFS vial upon coupling of the BFS vial relative to the delivery module to secure the BFS vial to the delivery module.

18. The pre-filled delivery assembly of claim 13 including a removable protective cover disposed over the administration member.

19. The pre-filled delivery assembly of claim 13 including a pivoting shield pivotally mounted relative to the connector body of the delivery module, the pivoting shield pivotal to selectively cover the administration member.

20. The pre-filled delivery assembly of claim 19 wherein the pivoting shield includes a latch to secure the pivoting shield to the administration member.

21. The pre-filled delivery assembly of claim 13 wherein the first and second fluid agents are different.
